(19) United States
(12) Patent Application Publication
(10) Pub. No.: US 2006/0275835 A1
(43) Pub. Date: Dec. 7, 2006

(54) Methods for identifying cell surface receptor protein modulators

(76) Inventors: Marlene A. Jacobson, Melrose Park, PA (US); Ruiping Wang, Maple Glen, PA (US)

Correspondence Address: MERCK AND CO., INC, P O BOX 2000, RAHWAY, NJ, 07065-0907, US

(21) Appl. No.: 10/563,692

(22) PCT Filed: Jul. 9, 2004
(86) PCT No.: PCT/US04/21889

§ 371 Date: Jan. 5, 2006

Related U.S. Application Data
(60) Provisional application No. 60/486,639, filed on Jul. 11, 2003.

(51) Int. Cl. G01N 33/567; G01N 33/53

(52) U.S. Cl. 435/7.2

(57) ABSTRACT

The present invention makes available a rapid, effective assay for screening and identifying pharmaceutically effective compounds that specifically interact with and modulate the activity of a target cell surface protein, e.g., a receptor or ion channel. The subject assay enables rapid screening of large numbers of compounds to identify those which modulate the bioactivity of the cellular proteins. The subject assays are particularly amenable for high throughput formats and are particular useful in identifying modulators of a mammalian metabotropic glutamate receptor protein.

### CROSS-REFERENCE TO RELATED APPLICATIONS

This application claims the benefit of U.S. Provisional Application No. 60/486,639, filed Jul. 11, 2003, the contents of which are incorporated herein by reference in their entirety.

### BACKGROUND OF THE INVENTION

In the mammalian central nervous system (CNS), the transmission of nerve impulses is controlled by the interaction between a neurotransmitter, that is released by a sending neuron, and a surface receptor on a receiving neuron, causing excitation of this receiving neuron. Of the approximately 20 naturally-occurring amino acids that are the basic building blocks for protein biosynthesis, certain amino acids, notably glutamate, are also used as signaling molecules in higher organisms such as man. In fact, glutamate, a member of a broad class of excitatory amino acids, is the transmitter of the vast majority of the excitatory synapses in the mammalian central nervous system (CNS) and plays an important role in a wide variety of CNS functions such as long-term potentiation (learning and memory), the development of synaptic plasticity, motor control, respiration, cardiovascular regulation, emotional states and sensory perception. (For review, see Hollmann and Heinemann (1994) Annul Rev. Neurosci. 17:31-108). Glutamate produces its effects on central neurons by binding to and thereby activating cell surface receptors. See Watkins & Evans, Ann. Rev. Pharmacol. Toxicol., 21, 165 (1981); Monaghan, Bridges, and Cotman, Ann. Rev. Pharmacol. Toxicol., 29, 365 (1989); Watkins, Krogsgaard-Larsen, and Honore, Trans. Pharm. Sci., 11, 25 (1990).

The cell surface receptors activated by glutamate have been subdivided into two major classes, the (i) ionotropic and (ii) metabotropic glutamate receptors, based on the structural features of the receptor proteins, the means by which the receptors transduce signals into the cell, and pharmacological profiles.

The “ionotropic” glutamate receptors (iGluRs) are ligand-gated ion channels that, upon binding glutamate, open to allow the selective influx of certain monovalent and divalent cations, thereby depolarizing the cell membrane. In addition, certain iGluRs with relatively high calcium permeability can activate a variety of calcium-dependent intracellular processes. These receptors are multisubunit protein complexes that may be homomeric or heteromeric in nature. The various iGluR subunits all share common structural motifs, including a relatively large amino-terminal extracellular domain (ECD), followed by two transmembrane domains (TMD), a second smaller extracellular domain, and a third TMD, before terminating with an intracellular carboxy-terminal domain.

The second general type of receptor is the G-protein or second messenger-linked “metabotropic” glutamate receptor. These receptors are coupled to multiple second messenger systems that activate a variety of intracellular second messenger systems following the binding of glutamate. Activation of mGluRs in intact mammalian neurons can elicit one or more of the following responses: activation of phospholipase C, increases in phosphoinositide (PI) hydrolysis, intracellular calcium release, activation of phospholipase D, activation or inhibition of adenylyl cyclase, increases or decreases in the formation of cyclic adenosine monophosphate (cAMP), activation of guanylyl cyclase, increases in the formation of cyclic guanosine monophosphate (cGMP), activation of phospholipase A₂, increases in arachidonic acid release, and increases or decreases in the activity of ion channels (e.g., voltage- and ligand-gated ion channels). Schoepp & Conn (1993), Trends Pharmacol. Sci. 14:13; Schoepp (1994), Neurochem. Int. 24:439; Pin & Duvoisin (1995), Neuropharmacology 34:1. Both types of receptors appear not only to mediate normal synaptic transmission along excitatory pathways, but also participate in the modification of synaptic connections during development and throughout life. Schoepp, Bockaert, and Sladeczek, Trends in Pharmacol. Sci., 11, 508 (1990); McDonald and Johnson, Brain Research Reviews, 15, 41 (1990).

Based on their amino acid sequence homology, agonist pharmacology, and coupling to transduction mechanisms, the 8 presently known mGluR sub-types are classified into three groups. Group I receptors (mGluR1 and mGluR5 and their alternatively spliced variants) have been shown to be coupled to stimulation of phospholipase C resulting in phosphoinositide hydrolysis and the subsequent mobilization of intracellular calcium. Masu et al. (1991), Nature 349:760; Pin et al. (1992), Proc. Natl. Acad. Sci. USA 89:10331, and, in some expression systems, to modulation of ion channels, such as K+ channels, Ca²⁺ channels, non-selective cation channels, or NIVIDA receptors. Group II receptors (mGluR2 and mGluR3) and Group III receptors (mGluRs 4, 6, 7, and 8) are negatively coupled to adenylylcyclase and have been shown to couple to inhibition of cAMP formation when heterologously expressed in mammalian cells, and to G-protein-activated inward rectifying potassium channels in Xenopus oocytes and in unipolar brush cells in the cerebellum. Nakanishi (1994), Neuron 13:1031; Pin & Duvoisin (1995), Neuropharmacology 34:1; Knopfel et al. (1995), J Med. Chem. 38:1417. The mGluR-mediated increase in intracellular Ca²⁺ concentration can activate Ca²⁺-sensitive K+ channels and Ca²⁺-dependent nonselective cationic channels. These mGluR-mediated effects often result from mobilization of Ca²⁺ from ryanodine sensitive, rather than Ins(1,4, 5)P3-sensitive, Ca2+ stores, suggesting that close functional interactions exist between mGluRs, intracellular Ca2+ stores and Ca2+-sensitive ion channels in the membrane.

All of the mGluRs are structurally similar, in that they are single subunit membrane proteins possessing a large amino-terminal ECD, followed by seven putative TMDs, and an intracellular carboxy-terminal domain of variable length. The amino acid homology between mGluRs within a given group is approximately 70%, but drops to about 40% between mGluRs in different groups. For mGluRs in the same group, this relatedness is roughly paralleled by similarities in signal transduction mechanisms and pharmacological characteristics.

Numerous important drugs for the treatment of various disease conditions act by influencing the activity of G-protein coupled receptors. Examples include agonist analogs of gonadotropin-releasing hormone, such as leuprolide, gonadorelin and nafarelin, which have been used to treat prostate and breast carcinomas, uterine leimyomatas, endometriosis, precocious puberty and nontumorous ovarian hyperandrogenic syndrome (see e.g., Pace, J. N. et al. (1992) Am. Fam. Physician 44:1777-1782), the cardiac βadrenergic receptor antagonist propranolol, which has been used to treat hypertension, angina pectoris and psychiatric disorders (see e.g., Nace, G. S. and Wood, A. J. (1987) Clin. Pharmacokinet. 13:51-64; Ananth, J. and Lin, K. M. (1986) Neuropsychobiology 15:20-27), the pulmonary □2-adrenergic receptor agonist metaproterenol, which has been used as a bronchodilator (see e.g., Hurst, A. (1973) Ann. Allergy 31:460-466) and the histamine 2 receptor antagonist cimetidine, which has been used to treat ulcers and idiopathic urticaria (see e.g., Sontag, S. et al. (1984) N. Engl. J Med. 311:689-693; Choy, M. and Middleton, R. K. (1991) DICP:609-612).

During the past twenty-five years, a revolution in understanding the basic structure and chemistry of the synaptic interconnections of neural tissues has taken place, which has yielded knowledge relevant to the neurotransmission. In the synapse, an axon terminal of a presynaptic cell contains vesicles filled with a neurotransmitter, such as glutamate which is released by exocytosis when a nerve impulse reaches the axon terminal. The vesicles release their contents into the synaptic cleft and the transmitter diffuses across the synaptic cleft. After a brief lag time (e.g., about 0.5 ms) the transmitter binds to receptors on postsynaptic cells. This typically causes a change in ion permeability and electrical potential in the postsynaptic cell.

Consequently, amino acids that function as neurotransmitters must be scavenged from the synaptic cleft between neurons to enable continuous repetitive synaptic transmission. For these reasons, specialized trans-membrane transporter proteins have evolved in all organisms to recover or scavenge extracellular amino acids (see Christensen, 1990, Physiol. Rev. 70:43-77 for review). For a review of neurotransmitter and transporter systems, see, Neurotransmitter Transporters: Structure, Function and Regulation (1997) M. E. A. Reith, ed. Human Press, Towata N.J., and the references cited therein. These transporters, which reduce intersynaptic concentration of neurotransmitters, are characteristically ion dependent, of high affinity, and are temperature sensitive. Nicholls & Attwell, 1990, TiPS 11: 462-468). In the case of glutamate, extracellular glutamate concentrations are maintained within physiological levels exclusively by glutamate transporters (GluTs), since no extracellular enzymes exist for the breakdown of glutamate (Robinson and Dowd, 1997). Consequently, GluTs are responsible for the high-affinity uptake of extracellular glutamate. They permit normal excitatory transmission as well as protection against excitotoxicity (Robinson and Dowd, 1997). A vast body of data suggests that high extracellular amino acid concentrations are associated with a number of pathological conditions, including ischemia, anoxia and hypoglycemia, as well as chronic illnesses such as Huntington's disease, Parkinson's disease, Alzheimer's disease, epilepsy and amyotrophic lateral sclerosis (ALS; see Pines et al., 1992, Nature 360: 464-467).

Metabotropic glutamate receptors have been suggested to play roles in a variety of pathophysiological processes and disease states affecting the CNS. These include stroke, head trauma, anoxic and ischemic injuries, hypoglycemia, epilepsy, anxiety, and neurodegenerative diseases such as Alzheimer's disease. Schoepp & Conn (1993), Trends Pharmacol. Sci. 14:13; Cunningham et al. (1994), Life Sci. 54:135; Hollman & Heinemann (1994), Ann. Rev. Neurosci. 17:31; Pin & Duvoisin (1995), Neuropharmacology 34:1; Knopfel et al. (1995), J. Med. Chem. 38:1417) pain (Salt and Binns (2000) Neurosci. 100:375-380, Bhave et al. (2001)Nature neurosci. 4:417-423), anxiety (Tatarczynska et al. (2001) Br. J. Pharmacol 132:1423-1430, Spooren et al. (2000) J. Pharmacol. Exp. Therapeut. 295:1267-1275), addiction to cocaine (Chiamulera et al. (2001)Nature Neurosci. 4:873-874), and schizophrenia (reviewed in Chavez-Noriega et al. (2002) Current Drug Targets:CNS & Neurological Disorders 1:261-281). Much of the pathology in these conditions is thought to be due to excessive glutamate-induced excitation of CNS neurons. Since Group I mGluRs appear to increase glutamate-mediated neuronal excitation via postsynaptic mechanisms and enhanced presynaptic glutamate release, their activation may contribute to the pathology. Therefore, selective antagonists of these receptors could be therapeutically beneficial, specifically as neuroprotective agents or anticonvulsants. In contrast, since activation of Group II and Group III mGluRs inhibits presynaptic glutamate release and the subsequent excitatory neurotransmission, selective agonists for these receptors might exhibit similar therapeutic utilities.

Metabotropic glutamate receptor agonists have been reported to have effects on various physiological activities. For example, trans-ACPD has been reported to possess both proconvulsant and anticonvulsant effects (Zheng and Gallagher, Neurosci. Lett. 125:147, 1991; Sacaan and Schoepp, Neurosci. Lett. 139:77, 1992; Taschenberger et al., Neuroreport 3:629, 1992; Sheardown, Neuroreport 3:916, 1992), and neuroprotective effects in vitro and in vivo (Pizzi et al., J. Neurochem. 61:683, 1993; Koh et al., Proc. Natl. Acad. Sci. USA 88:9431, 1991; Birrell et al., Neuropharmacol. 32:1351, 1993; Siliprandi et al., Eur. J. Pharmacol. 219:173, 1992; Chiamulera et al., Eur. J. Pharmacol. 216:335, 1992). The metabotropic glutamate receptor antagonist L-AP3 was shown to protect against hypoxic injury in vitro (Opitz and Reymann, Neuroreport 2:455, 1991).

However, a large body of evidence compels the conclusion that the currently available mGluR agonists and antagonists may be of limited use, both as research tools and potential therapeutic agents, as a result of their lack of potency and selectivity. Sacaan & Schoepp (1992), Neuro. Sci. Lett. 139:77; Lipparti et al. (1993), Life Sci. 52:85. But, other studies indicate that ACPD can inhibit epileptiform activity (Taschenberger et al. (1992), Neuroreport 3:629; Sheardown (1992), Neuroreport 3:916), and can also exhibit neuroprotective properties (Koh et al. (1991), Proc. Natl. Acad. Sci. USA 88:9431; Chiamulera et al. (1992), Eur. J. Pharmacol. 216:335; Siliprandi et al. (1992), Eur. J. Pharmacol. 219:173; Pizzi et al. (1993), J. Neurochem. 61:683.

The widespread expression of various metabotropic glutamate receptors and the lack of sufficiently selective mGluR agonists have been a major impediment to the successful development of direct-acting mGluR agonists to exploit the beneficial properties of mGluR. Consequently, other pharmacological approaches such as allosteric modulators of mGluR may prove to be a valuable alternative to direct-acting mGluR agonists and nucleoside uptake blockers. Thus, allosteric modulators of mGluR function should provide a more selective therapeutic effect than direct-acting mGluR agonists thereby decreasing systemic side effects attending conventional therapeutics.

The development of high through-put functional assays for GPCRs would greatly enhance the ability to discover and develop novel agonists and antagonists to this important super family of pharmaceutical targets. Indeed, with the advent of high-throughput functional assays, it has been possible to expand the search for pharmacological tools to include compounds that act on receptors at allosteric sites rather than at the historically targeted orthosteric sites. The first such compounds described for the mGluRs were MPEP and CPCCOEt, negative allosteric modulators selective for mGluR5 and mGluR1, respectively. Recently, positive allosteric modulators selective for mGluR1b also have been identified (Knoflach et al. 2001). For a review, refer to Conn and Pin (1997) and Schoepp et al. (1999). Other, modulatory effects expected of metabotropic glutamate receptor modulators include synaptic transmission, neuronal death, neuronal development, synaptic plasticity, spatial learning, olfactory memory, central control of cardiac activity, waking, control of movements, and control of vestibule ocular reflex (for reviews, see Nakanishi, Neuron 13:1031-37, 1994; Pin et al., Neuropharmacology 34:1, 1995; Knopfel et al., J. Med. Chem. 38:1417, 1995).

High-throughput screening allows a large number of molecules to be tested. For example, a large number of molecules can be tested individually using rapid automated techniques or in combination with using a combinatorial library of molecules. Individual compounds able to modulate a target receptor activity present in a combinatorial library can be obtained by purifying and retesting fractions of the combinatorial library. Thus, thousands to millions of molecules can be screened in a short period of time. Active molecules can be used as models to design additional molecules having equivalent or increased activity.

In the case of metabotriopic glutamate receptor modulators, high-throughput screening of chemical libraries using cells stably transfected with individual, cloned mGluRs may offer a promising approach to identify new lead compounds which are active on the individual receptor subtypes. Knopfel et al. (1995), J. Med. Chem. 38:1417. These lead compounds could serve as templates for extensive chemical modification studies to further improve potency, mGluR subtype selectivity, and important therapeutic characteristics such as bioavailability. Active molecules can be used as models to design additional molecules having equivalent or increased activity. Preferably, the activity of molecules in different cells may be tested to identify a metabotropic glutamate receptor agonist or metabotropic glutamate receptor antagonist molecule which mimics or blocks one or more activities of glutamate at a first type of metabotropic glutamate receptor

One approach for developing a high through-put functional GPCR assay is the use of reporter gene constructs. Reporter gene constructs couple transcriptional enhancers that are regulated by various intracellular second messengers with appropriate promoter and reporter gene elements to produce a surrogate signal transduction system responsive to signaling pathways activated by various hormone receptors (Deschamps, Science, 1985 230 :1174-7; Montminy, Proc. Nail. Acad Sci USA, 1986 83 :6682-6686; Angel, Cell, 1987, 49:729-39; Fisch, Mol. Cell Biol, 1989 9:1327-31). However, data generated by conventional high-throughput systems for measuring, for example, glutamate mediated signal transduction are contaminated by endogenous glutamate, which is produced and secreted from cultured cells. It is believed that this endogenous glutamate interferes with the ability to measure a true functional response of metabotropic glutamate receptors coupled to a reporter gene system. Specifically, the endogenous production of glutamate has been linked to high basal levels of reporter gene expression arising form activation of recombinantly expressed mGluR receptors by the endogenous glutamate.

While the mainstream of the pharmaceutical industry is moving to solve HTS throughput problems, e.g., by developing multi-well plates with more, and thus smaller, individual wells per plate, current models are still plagues by high-basal levels of reporter gene expression. This drawback is in addition to the expenditure of untold millions of dollars to achieve probably less than an order of magnitude increase in speed without other significant technological advantages which would increase the information content of the screening process.

Therefore there is a need for methods to assay the effects of compounds on the function of biological targets, exemplified by G-protein coupled receptors. In particular, there exists a need to identify modulators of metabotropic glutamate receptors for use in developing novel strategies for a variety of psychiatric and neurological disorders. It would be a further advancement to provide methods for screening for agonists, antagonists, and modulatory molecules that act on such receptors.

The present invention provides these and other features by providing a very sensitive assay system, which is adaptable to a high-throughput format.

In the main, the invention exploits the evolutionary principles responsible for the scavenging of amino acid neurotransmitters from the synaptic cleft between neurons together to create a cell-surface receptor based system capable of detecting and discriminating between thousands of second messenger signals.

The invention detailed herein provides, inter alia, method(s) of identifying target cell surface receptor modulating moieties characterized by a system wherein the indicator cells co-express a target receptor, vis-à-vis any one of a metabotropic glutamate receptor protein and a neurotransmitter transport protein specific for a ligand of said receptor, such as a murine glutamate transport protein, wherein the effect of the candidate agent can readily be determined using methods well known to one skilled in the art, e.g.; Ca²⁺ influx assay or a reporter gene based assay. Thus, the co-expression of a glutamate transporter, such as GLAST, in cells expressing any one of a metabotropic glutamate receptor effectively removes the endogenous extracellular glutamate from the media thereby allowing one to measure mGluR activation coupled to a reporter gene system, e.g., NFAT driven β-lactamase.

The method will find use for modeling transporter activity, transmitter degradation activity, in addition to the detection of modulators of cell surface receptors. These and many other features will be apparent upon complete review of the following disclosure.

### SUMMARY OF THE INVENTION

Given the important role of GPCRs, in both normal cellular responses and aberrant disease processes, assays that allow for the identification of agonists or antagonists of GPCRs are highly desirable.

As a non-limiting introduction to the breadth of the invention, the invention includes several general and useful aspects, including:

1) a method for identifying binding partners for G-protein coupled receptors.

2) a method for identifying candidate agents or compounds that directly or indirectly modulate (e.g. activate or inhibitor potentiate) a cell surface receptor such as a glutamate receptor with a reduced signal to noise ratio compared to the prior art assays.

3) the proposed assay(s) is very suitable for use in a high-throughput assay format.

In accordance with the above, the present invention provides functional assays for identifying pharmaceutically effective compounds that specifically interact with and modulate the activity of a target cell surface receptor of a cell. The methods of the invention will find use in identifying modulators. The compounds can be tested in these assays singly or, more preferably, in libraries of compounds, which effectively allows for rapid screening of large panels of compounds.

Receptor proteins for use in the present invention can be any receptor or ion channel which interacts with an extracellular molecule (i.e. hormone, growth factor, peptide, ion) to modulate a signal in the cell. To illustrate the receptor can be a cell surface receptor, e.g., a G-protein coupled receptor, such as a neurotransmitter receptor. Preferred G protein coupled receptors include any one or more members of the metabotropic glutamate receptor super family, exemplified by one or more of mGluRs 1 through 8.

The present invention provides for the use of any type of cell in the subject assays, whether prokaryotic or eukaryotic. In preferred embodiments, the cells of the present invention are eukaryotic. In certain preferred embodiments the cells are mammalian cells. The host cells can be derived from primary cells, or transformed and/or immortalized cell lines.

In the main, the assays of the invention provide a means for detecting the ability of one or more compounds to modulate the signal transduction activity of the target receptor protein by measuring at least one parameter of cellular metabolism of the receptor protein, e.g., up or down regulation of a detection signal. Thus, the binding event, e.g., interaction of a modulating moiety with the target receptor leads to the production of second messengers, such as cyclic AMP (e.g., by activation of adenylate cyclase), diacylglycerol or inositol phosphates, whose activation is ultimately detected.

On one hand, endogenous second messenger generation e.g., calcium mobilization or phospholipid hydrolysis or increased transcription of an endogenous gene can be detected directly. Alternatively, the use of a reporter or indicator gene can provide a convenient readout. By whatever means measured, a change, e.g., a statistically significant change in the detection signal can be used to facilitate isolation of those cells from the mixture which have received a signal via the target receptor, and thus can be used to identify novel compounds which function as receptor agonists or antagonists.

Where the signals generated are second messenger signals, these are well known to those of ordinary skill in the art. Such signals include those that cause alterations in calcium levels in the cell. Preferably, the signal detected is calcium mediated fluorescence. Such assays are well known to those of ordinary skill in the art. Where the signal is calcium mediated fluorescence, the cells can be virtually any cell known to those of ordinary skill in the art which have altered calcium levels as a result of the foregoing receptors. Fibroblasts, 3T3 cells, lymphocytes, keratinocytes, etc., may be used.

In yet other embodiment the invention provides a fluorescent ligand binding assay comprising: incubating cells with a fluorescent ligand capable of binding to cell surface receptors and measuring the fluorescence of cell bound ligand using FLIPR.

In one embodiment, the signal detected can be compared to a signal generated by a cell expressing a dysfunctional receptor protein or one that does not express a metabotropic glutamate receptor protein, or that has not been contacted with the modulating moiety thereby permitting the identification of a modulator mGluR protein activity.

In another method, the measurement of intracellular calcium can also be performed on a 96-well (or higher) format and with alternative calcium-sensitive indicators, preferred examples of these are: aequorin, Fluo-3, Fluo-4, Fluo-5, Calcium Green-1, Oregon Green, and 488 BAPTA. After activation of the receptors with agonist ligands the emission elicited by the change of intracellular calcium concentration can be measured by a luminometer, or a fluorescence imager; a preferred example of this is the fluorescence imager plate reader (FLIPR).

As noted above, the induction of a signal may also be measured by detecting the induction of a reporter gene (comprising a target-responsive regulatory element operatively linked to a nucleic acid encoding a detectable marker, e.g., luciferase) which is covalently linked to and co-expressed with the cell surface receptor protein encoding polynucleotide.

In accordance with the above, in one embodiment of the present invention the indicator cells express the receptor of interest endogenously. In other embodiments, the cells are engineered to express a heterologous receptor protein. In either of these embodiments, it may be desirable to co-express a neurotransmitter transport protein in the indicator cells. As well, other proteins involved in transducing signals from the target receptor can be complemented with an ortholog or paralog from another organism.

In one embodiment, the assays of the present invention can be used to screen compounds which are exogenously added to cells in order to identify potential receptor effector compounds. In another embodiment the subject assays may be used to rapidly screen large numbers of polypeptides in a library expressed in the cell in order to identify those polypeptides which agonize, antagonize or potentiate receptor bioactivity, thereby creating an autocrine system. The proposed autocrine assay is characterized by the use of a library of recombinant cells, wherein each cell includes a target receptor protein whose signal transduction activity can be modulated by interaction with an extracellular signal (modulating moiety), the transduction activity being able to generate a detectable signal, and an expressible recombinant gene encoding an exogenous test polypeptide from a polypeptide library. Preferably, the cell co-expresses a neurotransmitter transport protein specific for a ligand of said target cell surface receptor protein.

In another embodiment of the assay, if a test compound does not appear to directly induce the activity of the receptor protein, the assay may be repeated and modified by the introduction of a step in which the cell is first contacted with a known activator (agonist) of the target receptor to induce the signal transduction pathways from the receptor. Thus, a test compound can be assayed for its ability to antagonize, e.g., inhibit or block the activity of the activator. It is preferred that the assay include the step of contacting the indicator cell with the composition under investigation contemporaneously with a known agonist or the known agonist may be added or contacted just prior to the addition or contact with the test composition.

As well, the herein disclosed assay(s) may also be used to identify compounds which potentiate the induction response generated by treatment of the cell with a known activator. As used herein, an “agonist” refers to agents which either induce activation of receptor signaling pathways, e.g., such as by mimicking a ligand for the receptor, as well as agents which potentiate the sensitivity of the receptor to a ligand, e.g., lower the concentrations of ligand required to induce a particular level of receptor-dependent signaling, or increases or decreases of the affinity of the target receptor for its binding partner.

In other embodiments, the indicator/host cell harbors a reporter construct containing a reporter gene in operative linkage with one or more transcriptional regulatory elements responsive to the signal transducing activity of the receptor protein. Exemplary reporter genes include enzymes, such as luciferase, phosphatase, or β-galactosidase which can produce a spectrometrically active label, e.g., changes in color, fluorescence or luminescence. In preferred embodiments: the reporter gene encodes a gene product selected from the group consisting of β-lactamase chloramphenicol acetyl transferase, β-galactosidase and secreted alkaline phosphatase.

In its broadest aspect, the invention provides a method of identifying a modulating moiety of a GPCR protein, comprising: (a) contacting a population of indicator cells (mammalian cells/host cells) with a composition whose ability to modulate activity of said GPCR is sought to be determined; (b) measuring at least one parameter of cellular metabolism of the indicator cells; and (c) identifying at least one test compound as a modulator of the GPCR. Step b) may encompass monitoring said cells for a change in the level of a particular signal associated with activation of the target GPCR.

As used herein, the term “G-protein coupled receptor” (or “GPCR”) refers to a target receptor that, when expressed by a cell, associates with a G-protein (e. g., a protein which hydrolyzes GTP). Preferably, the GPCR is a “seven transmembrane segment receptor” (or “7 TMS receptor”), which refers to a protein that structurally comprises seven hydrophobic transmembrane spanning regions. Preferably, the G-protein is a member of the metabotropic glutamate receptor family.

As used herein, the term “population of indicator cells” “test cells” “reagent cells” refers to a plurality of cells wherein a cell co-expresses (1) at least one GPCR of interest (i.e., the GPCR for which a receptor modulator, e.g., agonist, antagonist or potentiator is to be identified) and (2) a neurotransmitter transport protein having affinity for a ligand specific for said GPCR. Thus, where the target receptor is a metabotropic glutamate receptor, the transport protein is preferably a glutamate transporter protein, preferably a mGLAST protein (mGLAST), and more preferably a murine glutamate transporter protein, having affinity for glutamate, a natural ligand of said metabotropic glutamate receptor protein. An indicator cell thus “co-expresses” a GPCR and a transporter protein, wherein the GPCR and the transport protein is present on a membrane of the indicator cells. The indicator cells may naturally express the GPCR of interest (also referred to as “endogenous” expression) or, more preferably, the indicator cells express the GPCR of interest because a nucleic acid molecule that encodes the receptor has been introduced into the indicator cells, thereby allowing for expression of the receptor on the membrane of the cells (also referred to as “exogenous” expression).

As used herein, the term “parameter of cellular metabolism” is intended to include detectable indicators of cellular responses that are regulated, at least in part, by a GPCR expressed by the indicator cell. Examples of parameters of cellular metabolism that can be measured or determined in the assays of the invention include second messengers produced as a result of the activation of the target receptor. A “test compound” or “composition under investigation” is identified as a modulating moiety which acts as a receptor agonist, antagonist or potentiator based upon its causing a change in at least one parameter of cellular metabolism of the indicator cells when the test compound is contacted with the indicator cells, as compared to the cellular metabolism of the indicator cells in the absence of the test compound or in the presence of indicator cells expressing a dysfunctional receptor protein. Typically, the compound either mimics one or more effects of glutamate at the metabotropic glutamate receptor, or blocks one or more effects of glutamate at the metabotropic glutamate receptor (or potentially both). Alternatively, the compound mimics one or more effects of glutamate at an allosteric site. The method can be carried out in vitro or in vivo.

The term “mimics” means that the compound causes a similar effect to be exhibited as is exhibited in response to contacting the receptor with glutamate. “Blocks” means that the presence of the compound prevents one or more of the normal effects of contacting the receptor with glutamate.

It is a further object of the present invention to provide compounds which selectively inhibit, activate modulate, or regulate metabotropic glutamate receptor subtypes.

It is also an object of the present invention to provide a method of selectively regulating glutamate reuptake.

In furtherance of a broad aspect, the invention encompasses a method for identifying compounds which modulate the activity of any one or more of the metabotropic glutamate receptor subtypes, comprising the steps of: a) contacting recombinant host cells, modified to contain the DNA of (i) a mammalian mGluR protein, which is operably linked to control sequences for expression whose activation can be coupled to Ca²⁺ signaling pathway, and (ii) a non-human neurotransmitter protein specific for a ligand of said receptor, with at least one compound or modulating moiety whose ability to modulate the activity of the mGluR is sought to be determined, and b) analyzing the cells for a difference in functional response mediated by said receptor. Preferably, the indicator cells are contacted or incubated with a known agonist of the target receptor protein prior to or contemporaneously with the compound or modulating moiety whose ability to modulate the activity of the target receptor is sought to be determined.

In one embodiment, step b) encompasses measuring the fluorescence of the test population from a calcium-sensitive fluorescent dye in a fluorometric imaging plate reader (FLIPR) thereby obtaining a first value. The fluorescence measurement is thereafter compared with a fluorescence measurement of a control mixture obtained by contacting an un-transformed form of the cell, e.g., not expressing a receptor protein or expressing a dysfunctional receptor protein with the same at least one candidate agent to obtain a second value. Where the first value is greater than the second, the candidate agent is an activator, while if the second value if greater than the first, then the candidate agent is an inhibitor of activation of the expressed receptor protein.

The assays of the invention are particularly suitable for an HTS format, which allows the proposed HTS format to test the action of a drug candidate upon a group of cells. The novel HTS system of the present invention can provide improved efficiency over current HTS methods since the vast majority of the cells produce endogenous glutamate which, in turn, effectively interferes with the end result. The presence of the glutamate transporter effective eliminates or quenches endogenous glutamate produced by the cell, thereby effectively reducing the signal to noise ratio and improving the overall sensitivity of the assay.

Methods to assay compounds to determine their cell receptor agonist or antagonist activity are also provided comprising determining the level of the transcriptional and/or translational products of the reporter gene which is produced when a recombinant cell of the present invention is contacted with media containing a compound to be tested. This level is then compared to the level of transcriptional and/or translational products of the reporter gene which is produced when cells of the recombinant cells are contacted with control media not containing the compound to be tested. Agonists of the cell receptor are identified as compounds which cause an increase in the level of transcriptional and/or translational products of the reporter gene as compared to cells not exposed to the compound. Antagonists of the cell receptor are identified as compounds which cause a decrease in the level of transcriptional and/or translational products of the reporter gene in agonist activated cells, as compared to agonist activated cells not exposed to the compound. Alternatively, levels of transcriptional and/or translational products of the reporter in the presence of a potential agonist or antagonist can be compared in cells expressing the receptor on their surface and cells which do not express the receptors on their surface.

In furtherance of the above object, an aspect of the invention provides a process for determining the modulating effect of a modulating moiety on a receptor mediated signal transmission pathway in a suitable host cell, e.g., a human or animal cell via the measurement of a reporter gene product. This process is characterized in that the modulating effect of the modulating moiety on a component in the signal transmission pathway initiated by activation of a metabotropic glutamate receptor is determined by incubating indicator cells with the test substance, and measuring the concentration of a reporter gene product relative to normal as indicative of the activation of the specific mGluR subtype.

The inventive system may be employed to detect reporter gene expression in any of a variety of contexts. For example, the reporter gene may be expressed in vivo or in vitro. In preferred embodiments of the invention, reporter gene expression is monitored in a high-throughput format. The assay system therefore allows analysis of large numbers of compounds that may alter or affect expression of the reporter gene. In certain preferred embodiments, the collection of compounds assayed represents at least a portion of a combinatorial library. The inventive assay system may also take advantage of other technological advances in high-throughput screening, including robotic machines, microarrayers and other arraying devices, high-density plates, fluorescence-activated bead sorting (FABS), CCD cameras, microscopes, fluorescence microscopy, and computer analysis.

Yet another aspect of the invention, features a method of screening for a compound that binds to one or more metabotropic glutamate receptor subtypes. The method aims to detect binding based upon the induction of a second messenger response. The method involves introducing into a cell one or more metabotropic glutamate receptors and a glutamate transporter protein to form an indicator cell medium and incubating a test compound and said cell population into an acceptable medium, which includes a known agonist of said glutamate receptor subtype and monitoring the binding of the test compound to said receptor by analyzing the cells for a difference in functional response mediated by the interaction of the test compound and the respective metabotropic glutamate receptor protein.

Compounds targeted to one or more metabotropic glutamate receptor proteins can have several uses including therapeutic uses and diagnostic uses. Those compounds binding to a metabotropic glutamate receptor and those compounds efficacious in modulating metabotropic receptor glutamate activity can be identified using the procedures described herein. Those compounds which can selectively bind to the metabotropic glutamate receptor can be used therapeutically, or alternatively as diagnostics to determine the presence of the metabotropic glutamate receptor versus other glutamate receptors.

As well, a compound determined by a process according to the invention and a composition, for example, a pharmaceutical composition, which comprises an effective amount of a mammalian metabotropic glutamate receptor agonist determined to be such by a process according to the invention, and a carrier, for example, a pharmaceutically acceptable carrier are also encompassed by the invention.

These aspects of the invention, as well as others described herein, can be achieved by using the methods and compositions of matter described herein. To gain a full appreciation of the scope of the invention, it will be further recognized that various aspects of the invention can be combined to make desirable embodiments of the invention. For example, the invention includes a method of identifying compounds that modulate active genomic polynucleotides operably linked to a protein with β-lactamase activity that can be detected by FACS using a fluorescent, membrane permeant β-lactamase substrate. Such combinations result in particularly useful and robust embodiments of the invention.

### BRIEF DESCRIPTION OF THE DRAWINGS

FIG. 1: Quisqualate, glutamate and 3,5-DHPG activate Ca²⁺ transients in human mGluR5/mGLAST expressing CHONFAT cells: Human mGluR5 CHONFAT/mGLAST cells were plated in clear-bottomed 384 well plates in glutamate/glutamine-free medium, loaded the next day with the calcium-sensitive fluorescent dye Fluo-4, and placed in FLIPR₃₈₄. Agonists were added after 10 seconds of baseline determination. Data were normalized to a glutamate (10 μM) control maximum. Concentration-response curves were generated from mean data of 5 experiments. Error bars are SEM. EC₅₀ values for these cells are given in the figure.

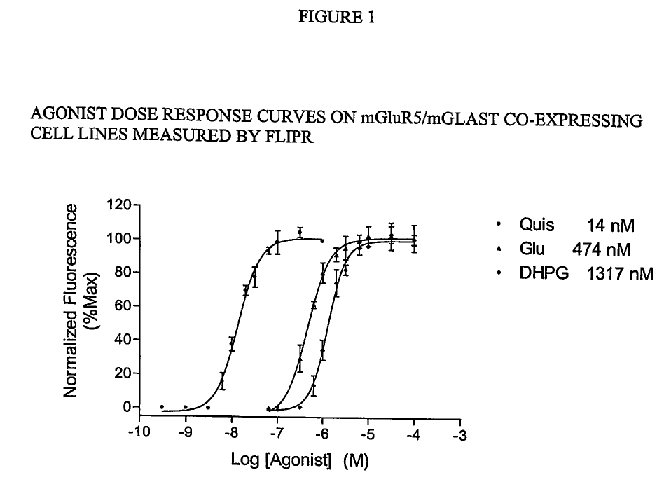

FIG. 2: Effect of mGLAST expression in mGluR5CHONFAT cells on basal activity of βlactamase gene reporter. Panel 1 (top) compares the fluorescence ratio (EM460/530) in non-transfected CHONFAT cells (0.2), CHONFAT cells stimulated with 200 nM thapsigargin (1.0; maximal fluorescence), mGluR5CHONFAT cells without mGLAST co-expression (0.9), in the absence of exogenously added agonist and mGluR5CHONFAT without mGLAST co-expression+1 μM quisqualate (0.85). High backgrounds observed without mGluR5 agonist is due to endogenous glutamate produced by the cells. Panel 2 (bottom) compares EM460/530 ratios on mGluR5CHONFAT cells without and with mGLAST co-expression. mGluR5CHONFAT+mGLAST EM460/530=0.18; mGluR5CHONFAT-GLAST EM460/530=0.9; mGluR5CHONFAT+Mglast=10 μM quisqualate=1.35. The co-expression of mGLAST in the mGluR5CHONFAT cells decreased the background by eliminating endogenous glutamate and allows for the measurement of receptor activation by exogenously added agonists.

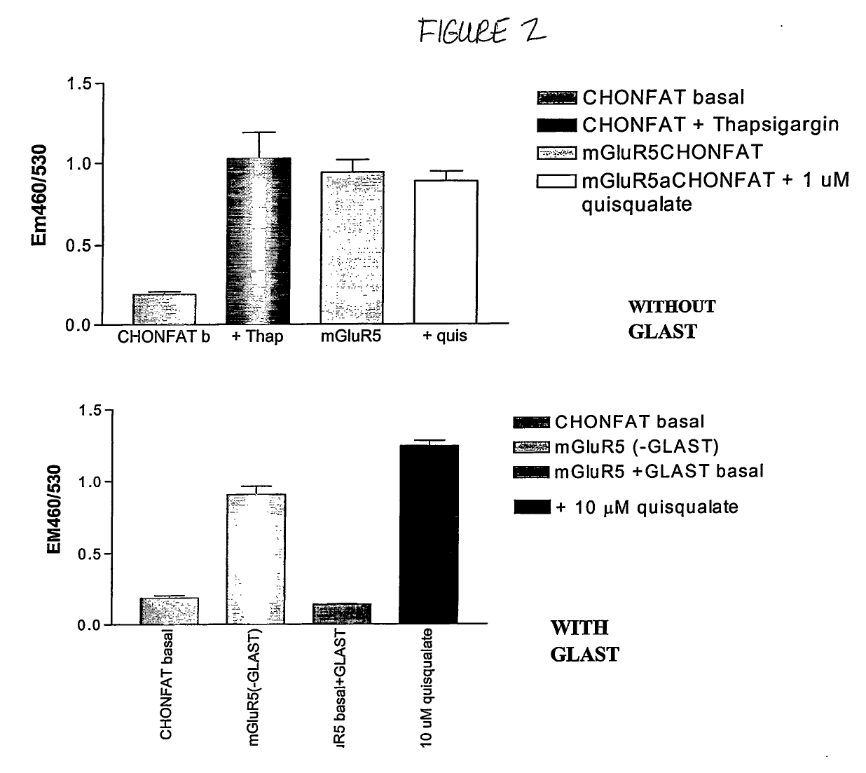

FIG. 3: β-lactamase reporter gene assay in mGluR5CHONFAT co-expressing mGLAST.

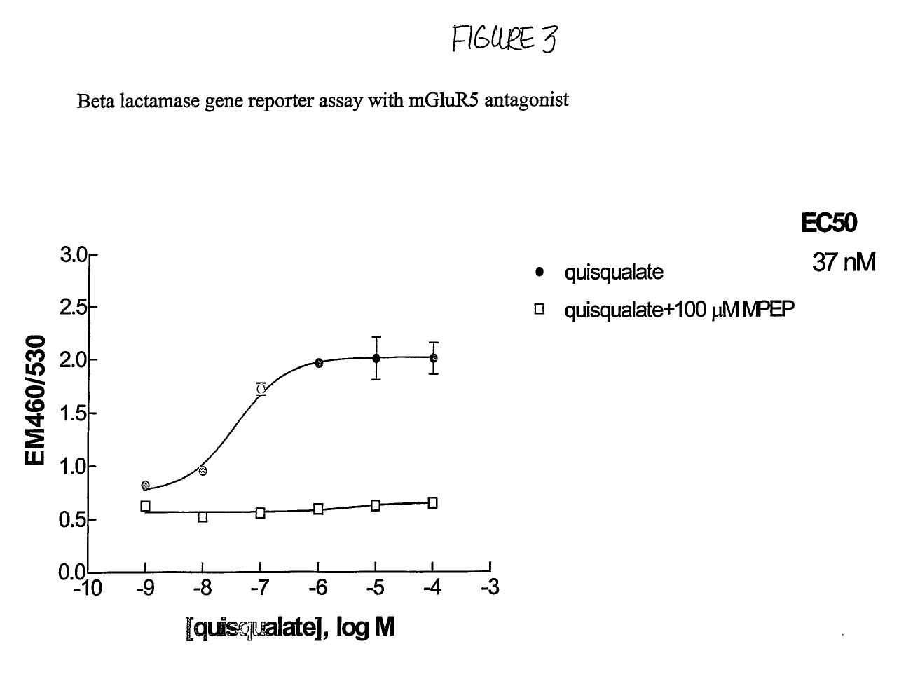

Dose response of the mGluR5 agonist quisqualate in the absence (EC50=44 nM) and presence (EC50=15 nM) of 10 μM positive allosteric modulator.

FIG. 4: Assay of mGluR5 antagonist, MPEP, in the β-lactamase reporter gene assay in mGluR5CHONFAT co-expressing mGLAST. MPEP was pre-incubated for 10 minutes before the addition of quisqualate.

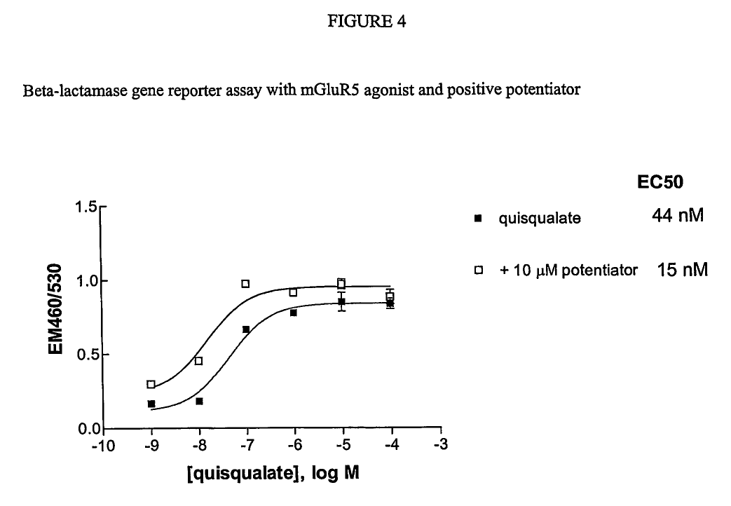

### DETAILED DESCRIPTION OF THE INVENTION

The identification of biological activity in new molecules has historically been accomplished through the use of in vitro assays or whole animals. Intact biological entities, either cells or whole organisms, have been used to screen for anti-bacterial, anti-fungal, anti-parasitic and anti-viral agents in vitro. Cultured mammalian cells have also been used in screens designed to detect potential therapeutic compounds. A variety of bioassay endpoints have been exploited in cell screens including the activation of a signal transduction pathway. For example, cytotoxic compounds used in cancer chemotherapy have been identified through their ability to inhibit the growth of tumor cells in vitro and in vivo. In vitro testing is a preferred methodology in that it permits the design of high-throughput screens wherein small quantities of large numbers of compounds can be tested in a short period of time and at low expense. Optimally, animals are reserved for the latter stages of compound evaluation and are not used in the discovery phase; the use of whole animals is labor-intensive and extremely expensive.

The heterologous expression of recombinant mammalian G protein-coupled receptors in mammalian cells which do not normally express those receptors has been described as a means of studying receptor function for the purpose of identifying agonists and antagonists of those receptors. Consequently, the search for modulators of cell surface receptors, e.g., agonists, antagonists and potentiators has been an intense area of research aimed at drug discovery due to the elegant specificity of these molecular targets.

The assay(s) of the present invention provide a convenient format for discovering drugs which can be useful to modulate cellular function, as well as to understand the pharmacology of compounds that specifically interact with cellular receptors or ion channels. Moreover, the subject assay is particularly amenable to identifying modulating moieties, natural or artificial, for receptors and ion channels.

The subject assay is useful for identifying modulating moieties (synthetic or biological) that interact with any receptor protein whose activity ultimately induces a signal transduction cascade in the host cell which can be exploited to produce a detectable signal. In particular, the assays can be used to test functional ligand-receptor or ligand-ion channel interactions for cell surface-localized receptors and channels. As described in more detail below, the subject assay are particularly used to identify effectors of, for example, G protein-coupled receptors, ion channels, and cytokine receptors. In preferred embodiments the method described herein is used for identifying modulating moieties for mammalian, more preferably human metabotropic glutamate receptor subtypes wherein the transport protein is from a non-human source and in particular it is a murine glutamate transport protein.

Before the present proteins, nucleotide sequences, and methods are described, it is to be understood that the present invention is not limited to the particular methodologies, protocols, cell lines, vectors, and reagents described, as these may vary. It is also understood that the terminology used herein is for the purpose of describing particular embodiments only, and is not to limit the scope of the present invention.

The singular forms “a,” “an,” and “the” include plural reference unless the context clearly dictates otherwise.

All technical and scientific terms used herein have the same meanings as commonly understood by one of ordinary skill in the art to which this invention pertains. The practice of the present invention will employ, unless otherwise indicated, conventional techniques of protein chemistry and biochemistry, molecular biology, microbiology and recombinant DNA technology, which are within the skill of the art. Such techniques are explained fully in the literature.

Although any machines, materials, and methods similar or equivalent to those described herein can be used to practice or test the present invention, the preferred machines, materials, and methods are now described. All patents, patent applications, and publications mentioned herein, whether supra or infra, are each incorporated by reference in its entirety.

Glossary

Before further description of the invention, certain terms employed in the specification, examples and appended claims are, for convenience, collected here.

A “gene” “oligonucleotide” or grammatical equivalents thereof refers to a nucleic acid molecule whose nucleotide sequence codes for a polypeptide molecule. Genes may be uninterrupted sequences of nucleotides or they may include such intervening segments as introns, promoter regions, splicing sites and repetitive sequences. A gene can be either RNA or DNA. A preferred gene is one that encodes the invention protein.

As used herein, “recombinant cells” include any cells that have been modified by the introduction of heterologous oligonucleotides, e.g., DNA. The terms “recombinant protein”, “heterologous protein” and “exogenous protein” are used interchangeably throughout the specification and refer to a polypeptide which is produced by recombinant DNA techniques, wherein generally, DNA encoding the polypeptide is inserted into a suitable expression vector which is in turn used to transform a host cell to produce the heterologous protein. That is, the polypeptide is expressed from a heterologous nucleic acid.

As used herein, the “activity” of a target cell surface receptor refers to the function of the receptor in mediating a cellular response to an extracellular signal. The “activity” of a receptor is reflected in the signaling function or the activity of downstream signaling pathways, or ultimately, in changes in the expression of one or more genes.

“Agonist” refers to a molecule which modulates the activity of the target cell surface receptor by, for example, increasing or prolonging the duration of the effect of the target receptor. Agonists for a particular receptor can be identified by contacting cells that co-expresses a particular receptor with a test molecule, and determining if that test molecule induces a response mediated by that particular receptor in a manner specific for that receptor.

“Antagonist” refers to a molecule which, when bound to the target receptor or within close proximity, decreases the amount or the duration of the biological activity of the receptor.

In general, the assays of the invention makes use of heterologous expression systems utilizing appropriate host cells to express the target cell surface receptor and a transport protein to obtain the desired second messenger coupling.

The term “receptor” denotes a cell-surface protein that binds to a bioactive molecule (i.e., a ligand) and mediates the effect of the ligand on the cell. Membrane-bound receptors are characterized by a multi-domain structure comprising an extracellular ligand-binding domain and an intracellular effector domain that is typically involved in signal transduction. Binding of ligand to receptor results in a conformational change in the receptor that causes an interaction between the effector domain and other molecule(s) in the cell. This interaction in turn leads to an alteration in the metabolism of the cell. Metabolic events that are linked to receptor-ligand interactions include gene transcription, phosphorylation, dephosphorylation, increases in cyclic AMP production, mobilization of cellular calcium, mobilization of membrane lipids, cell adhesion, hydrolysis of inositol lipids and hydrolysis of phospholipids

As used herein, the term “human metabotropic glutamate receptor activity” refers to the initiation or propagation of signaling by a human metabotropic glutamate receptor polypeptide. Human metabotropic glutamate receptor signaling activity is monitored by measuring a detectable step in a signaling cascade by assaying one or more of the following: stimulation of GDP for GTP exchange on a G protein; alteration of adenylate cyclase activity; protein kinase C modulation; phosphatidylinositol breakdown (generating second messengers diacylglycerol, and inositol triphosphate); intracellular calcium flux; modulation of tyrosine kinases; or modulation of gene or reporter gene activity. A detectable step in a signaling cascade is considered initiated or mediated if the measurable activity is altered by 10% or more above or below a baseline established in the substantial absence of glutamate relative to any of the human metabotropic glutamate receptor activity assays described herein below. Thus, in the case of a G-protein coupled receptor (GPCR), the activity of the GPCR such as the mammalian metabotropic glutamate receptor polypeptides may be measured using any of a variety of functional assays in which activation of the receptor in question results in an observable change in the level of some second messenger system. In various embodiments change(s) in the level of an intracellular second messenger responsive to signaling by a cell surface receptor are detected. For example, in various embodiments the assay may assess the ability of test agent to cause changes in adenylate cyclase activity (cAMP production), GTP hydrolysis, calcium mobilization, arachidonic acid release, ion channel activity, inositol phospholipid hydrolysis (IP₃, DAG production) or guanylyl cyclase upon receptor stimulation. By detecting changes in intracellular signals, such as alterations in second messengers or gene expression in cells candidate agonists and antagonists to the cell surface receptor-dependent signaling can be identified. Alternatively, the measurable activity can be measured indirectly, as in, for example, a reporter gene assay. Any one or more subtypes of metabotropic glutamate receptor can be assayed using the methods provided herein. Suitable metabotropic glutamate receptor include at least one selected from the group consisting of mGluR-1, -2, -3, -4, -5, -6, -7, and -8. The invention also embraces isolated functionally equivalent variants, useful analogs and fragments of the foregoing metabotropic glutamate receptor subtypes and nucleic acid molecules encoding same including molecules which selectively bind an antibody specific for any one or more of the metabotropic glutamate receptor proteins.

As used herein, the term “parameter of cellular metabolism” is intended to include detectable indicators of cellular responses that are regulated, at least in part, by a GPCR expressed by the indicator cell. Examples of parameters of cellular metabolism that can be measured or determined in the assays of the invention include second messengers produced as a result of the activation of the target receptor.

As used herein, the term “second messenger” refers to a molecule, generated or caused to vary in concentration by the activation of a G-Protein Coupled Receptor that participates in the transduction of a signal from that GPCR Non-limiting examples of second messengers include cAMP, diacylglycerol, inositol triphosphates and intracellular calcium. The term “change in the level of a second messenger” refers to an increase or decrease of at least 10% in the detected level of a given second messenger relative to the amount detected in an assay performed in the absence of a candidate modulator.

The terms “background” or “background signal intensity” refer to signals resulting from endogenous glutamate produced by the cells under investigation. A single background signal can also be calculated for each target cell population. Alternatively, background may be calculated as the average signal intensity produced by target cell prior to the step of contacting or incubating said cells with a modulating moiety e.g., glutamate.

The term “test chemical” refers to a chemical to be tested by one or more method(s) of the invention as a putative modulator. A test chemical is usually not known to bind to the target of interest. The term “control test chemical” refers to a chemical known to bind to the target (e.g., a known agonist, antagonist, partial agonist or inverse agonist). A “test compound” or “composition under investigation” is identified as a modulating moiety which acts as a receptor agonist, antagonist or potentiator based upon its causing a change in at least one parameter of cellular metabolism of the indicator cells when the test compound is contacted with the indicator cells, as compared to the cellular metabolism of the indicator cells in the absence of the test compound or in the presence of indicator cells expressing a dysfunctional receptor protein. Typically, the compound either mimics one or more effects of glutamate at the metabotropic glutamate receptor, or blocks one or more effects of glutamate at the metabotropic glutamate receptor (or potentially both). Alternatively, the compound mimics one or more effects of glutamate at an allosteric site. The term “test chemical” does not typically include chemicals known to be unsuitable for a therapeutic use for a particular indication due to toxicity of the subject.

The term “mimics” means that the compound causes a similar effect to be exhibited as is exhibited in response to contacting the receptor with glutamate. “Blocks” means that the presence of the compound prevents one or more of the normal effects of contacting the receptor with glutamate.

As used herein, the term “detectable step” refers to a step that can be measured, either directly, e.g., by measurement of a second messenger or detection of a modified (e.g., phosphorylated) protein, or indirectly, e.g., by monitoring a downstream effect of that step. For example, adenylate cyclase activation results in the generation of cAMP. The activity of adenylate cyclase can be measured directly, e.g., by an assay that monitors the production of cAMP in the assay, or indirectly, by measurement of actual levels of cAMP. Likewise, as detailed infra, the mobilization of intracellular calcium or the influx of calcium from outside the cell may be a response to activation of the mGluR protein or lack thereof. Calcium flux in the indicator cell can be measured using standard techniques. The choice of the appropriate calcium indicator, fluorescent, bioluminescent, metallochromic, or Ca²⁺-sensitive microelectrodes depends on the cell type and the magnitude and time constant of the event under study (Borle (1990) Environ Health Perspect 84:45-56). As an exemplary method of Ca²⁺ detection, cells could be loaded with the Ca²⁺ sensitive fluorescent dye fura-2 or indo-1, using standard methods, and any change in Ca²⁺ measured using a fluorometer.

As used herein, the term “standard” refers to a sample taken from an individual who is not affected by a disease or disorder characterized by dysregulation of human metabotropic glutamate receptor or glutamate activity. The “standard” is used as a reference for the comparison of human metabotropic glutamate receptor or glutamate or mRNA levels and quality (i.e., mutant vs. wild-type), as well as for the comparison of human metabotropic glutamate receptor activities.

The term “modulate” refers to a change in the activity of a target cell receptor. For example, modulation may cause an increase or a decrease in protein activity, binding characteristics, or any other biological, functional, or immunological properties of a target cell receptor. The ability to modulate the activity of the target cell receptor can be exploited in assays to screen for organic, inorganic, or biological compounds which affect the above properties of a target cell receptor such as a mammalian metabotropic glutamate receptor.

A promoter is considered to be “modulated” by an active, promiscuous Gα protein when the expression of a reporter gene to which the promoter is operably linked is either increased or decreased upon activation of the promiscuous Gα protein. It is not necessary that the active, promiscuous Gα protein directly modulate reporter gene expression.

The phrases “percent identity” and “% identity” refers to the percentage of sequence similarity found by a comparison or alignment of two or more amino acid or nucleic acid sequences. Percent similarity can be determined by methods well-known in the art. Percent identity can be determined by a direct comparison of the sequence information between two molecules by aligning the sequences, counting the exact number of matches between the two aligned sequences, dividing by the length of the shorter sequence, and multiplying the result by 100. For example, percent similarity between amino acid sequences can be calculated using the cluster method. See, e.g., Higgins & Sharp, 73 GENE 237-44 (1988). The cluster algorithm groups sequences into clusters by examining the distances between all pairs. The clusters are aligned pairwise and then in groups. The percentage similarity between two amino acid sequences, e.g., sequence A and sequence B, is calculated by dividing the length of sequence A, minus the number of gap residues in sequence A, minus the number of gap residues in sequence B, into the sum of the residue matches between sequence A and sequence B, times one hundred. Gaps of low or of no homology between the two amino acid sequences are not included in determining percentage similarity. Percent similarity can be calculated by other methods known in the art, for example, by varying hybridization conditions, and can be calculated electronically using programs such as the MEGALIGN™ program (DNASTAR Inc., Madison, Wis.). Readily available computer programs can be used to aid in the analysis, such as ALIGN, Dayhoff, M. O. in Atlas of Protein Sequence and Structure M.O. Dayhoff ed., 5 Suppl. 3:353-358, National Biomedical Research Foundation, Washington, D.C., which adapts the local homology algorithm of Smith and Waterman (1981) Advances in Appl. Math. 2:482-489, for peptide analysis. Programs for determining nucleotide sequence identity are available in the Wisconsin Sequence Analysis Package, Version 8 (Genetics Computer Group, Madison, Wis.) for example, the BLAST, BESTFIT, FASTA, and GAP programs, which also rely on the Smith and Waterman algorithm. These programs are readily utilized with the default parameters recommended by the manufacturer and described in the Wisconsin Sequence Analysis Package referred to above. Other programs for calculating identity or similarity between sequences are known in the art.

As used herein, “functional equivalent” refers to a protein or nucleic acid molecule that possesses functional or structural characteristics that are substantially similar to a heterologous protein, polypeptide, enzyme, or nucleic acid of interest, e.g., mGluR family of GPCR's. A functional equivalent of a protein may contain modifications depending on the necessity of such modifications for the performance of a specific function. The term “functional equivalent” is intended to include the “fragments,” “mutants,” “hybrids,” “variants,” “analogs,” or “chemical derivatives” of a molecule.

Variant: an amino acid sequence that is altered by one or more amino acids. The variant may have “conservative” changes, wherein a substituted amino acid has similar structural or chemical properties, e.g., replacement of leucine with isoleucine. More rarely, a variant may have “nonconservative” changes, e.g., replacement of a glycine with a tryptophan. Analogous minor variations may also include amino acid deletions or insertions, or both. Guidance in determining which amino acid residues may be substituted, inserted, or deleted may be found using computer programs well known in the art, for example, DNASTAR.RTM. software.

The term “sample” is used in its broadest sense. A sample suspected of containing nucleic acids encoding one or more human metabotropic glutamate receptor protein subtypes, or fragments thereof, or an mGluR subtype polypeptide may comprise a bodily fluid; an extract from a cell chromosome, organelle, or membrane isolated from a cell; an intact cell; genomic DNA, RNA, or cDNA, in solution or bound to a substrate; a tissue; a tissue print; etc.

The term “transformed” refers to any known method for the insertion of foreign DNA or RNA sequences into a host prokaryotic cell. The term “transfected” refers to any known method for the insertion of foreign DNA or RNA sequences into a host eukaryotic cell. Such transformed or transfected cells include stably transformed or transfected cells in which the inserted DNA is rendered capable of replication in the host cell. They also include transiently expressing cells which express the inserted DNA or RNA for limited periods of time. The transformation or transfection procedure depends on the host cell being transformed. It can include packaging the polynucleotide in a virus as well as direct uptake of the polynucleotide, such as, for example, lipofection or microinjection. Transformation and transfection can result in incorporation of the inserted DNA into the genome of the host cell or the maintenance of the inserted DNA within the host cell in plasmid form. Methods of transformation are well known in the art and include, but are not limited to, viral infection, electroporation, lipofection, and calcium phosphate mediated direct uptake.

“Transporter protein(s)” regulate many different functions of a cell, including cell proliferation, differentiation, and signaling processes, by regulating the flow of molecules such as ions and macromolecules, into and out of cells. Transporters are found in the plasma membranes of virtually every cell in eukaryotic organisms. Transporters mediate a variety of cellular functions including regulation of membrane potentials and absorption and secretion of molecules and ion across cell membrane See Greger, R. (1988) Annu. Rev. Physiol. 50:111-122.

I. Overview of Assay

As set out herein, the present invention relates to methods for identifying effectors of a receptor protein or complex thereof. In its broadest sense, the invention provides methods for identifying G-protein coupled receptor (GPCR) agonists or antagonists, i.e., screening assays for agents that stimulate or inhibit the activity of a target GPCR. The methods of the invention are functional assays. The methods are based, at least in part, on the discovery of detectable changes in cellular metabolism that occur in indicator cells expressing a target GPCR when the indicator cells are contacted with a receptor modulator In general, the assays of the invention are characterized by the use of a library of recombinant cells, wherein each cell expresses (i) a recombinant gene encoding an exogenous target receptor protein whose signal transduction activity can be modulated by interaction with an extracellular signal, the transduction activity being able to generate a detectable signal, and (ii) an expressible transporter protein specific for a ligand of the receptor protein. The ability of particular constituents of the test compound library to modulate the signal transduction activity of the target receptor can be scored for by detecting up or down-regulation of the detection signal. For example, second messenger generation (e.g. calcium influx, GTPase activity, adenylyl cyclase activity or phospholipid hydrolysis) via the activation of a target receptor can be measured directly. Alternatively, the use of a reporter gene can provide a convenient readout. In any event, a statistically significant change in the detection signal can be used to facilitate identification of modulating moiety which is an effector of the target receptor.

By a method of the invention, a modulating moiety, which induce the receptor's signaling can be screened. If the test compound does not appear to induce the activity of the receptor protein, the assay may be repeated and modified by the introduction of a step in which the recombinant cell is first contacted with a known activator of the target receptor to induce signal transduction from the receptor, and the test modulating moiety is assayed for its ability to inhibit the activity of the receptor, e.g., to identify receptor antagonists.

In yet other embodiments, the compound/compound library can be screened for members which potentiate the response to a known activator of the receptor. In this respect, potential compounds can be identified by the present assay by testing their ability to potentiate the signal transduction in the presence and absence of a threshold amount of a known agonist.

Likewise, agonists can be identified by testing the compound in the presents of a target receptor co-expressing at least one metabotropic glutamate receptor protein subtype and a transport protein specific for a ligand of said receptor and testing the same in the cells expressing a dysfunctional receptor protein on those that do not express the receptor protein. This way, further compound libraries may be screened for members which potentiate, activate or inhibit the target receptor peptide.

Signal transduction via activation of a target cell surface receptor can also be detected via the use of a reporter gene based assay. To illustrate, the intracellular signal that is transduced can be initiated by the specific interaction of an extracellular signal, particularly a ligand, with a cell surface receptor on the cell. This interaction sets in motion a cascade of intracellular events, the ultimate consequence of which is a rapid and detectable change in the transcription or translation of a gene. By selecting transcriptional regulatory sequences that are responsive to the transduced intracellular signals and operatively linking the selected promoters to reporter genes, whose transcription, translation or ultimate activity is readily detectable and measurable, the transcription based assay provides a rapid indication of whether a specific receptor or ion channel interacts with a test peptide in any way that influences intracellular transduction. Expression of the reporter gene, thus, provides a valuable screening tool for the development of compounds that act as agonists or antagonists of a cell receptor or ion channel

Reporter gene based assays of this invention measure the end stage of the above described cascade of events, e.g., transcriptional modulation. Accordingly, in practicing one embodiment of the assay, a reporter gene construct is inserted into the reagent cell in order to generate a detection signal dependent on receptor signaling. Typically, the reporter gene construct will include a reporter gene in operative linkage with one or more transcriptional regulatory elements responsive to the signal transduction activity of the target receptor, with the level of expression of the reporter gene providing the receptor-dependent detection signal. The amount of transcription from the reporter gene may be measured using any method known to those of skill in the art to be suitable. In preferred embodiments, the gene product of the reporter is detected by an intrinsic activity associated with that product. For instance, the reporter gene may encode a gene product that, by enzymatic activity, gives rise to a detection signal based on color, fluorescence, or luminescence. The amount of expression from the reporter gene is then compared to the amount of expression in either the same cell in the absence of the test compound or it may be compared with the amount of transcription in a substantially identical cell that lacks the specific receptors.

In general, the assay(s) is characterized by the use of a mixture of cells to sample a battery of compounds for receptor/channel agonists or antagonists. As described with greater detail below, the indicator cells express a target receptor protein or ion channel capable of transducing a detectable signal in the indicator (test cells) with the proviso that the indicator cells, in addition to expressing the target cell surface receptor also express a transport protein specific for a ligand of the cell surface receptor protein.

With respect to the target receptor, it may be endogenously expressed by the host cell, or it may be expressed from a heterologous gene that has been introduced into the cell. Methods for introducing heterologous DNA into eukaryotic cells are of course well known in the art and any such method may be used. In addition, DNA encoding various receptor proteins is known to those of skill in the art or it may be cloned by any method known to those of skill in the art. In certain embodiments, such as when an exogenous receptor is expressed, it may be desirable to inactivate, such as by deletion, a homologous receptor present in the cell. The same holds true for the transport protein.

In preferred embodiments, the test compound (modulating moiety) is exogenously added, and its ability to modulate the activity of the target receptor is scored in the assay. However, in some embodiments, the modulating moiety may be a peptide endogenously produced by the same cell that express the target receptor and the transport protein thereby providing an autocrine cell and used to screen for those that activate, inhibit or potentiate the receptor protein Thus, in those embodiments, the assay provides a population of cells which express a library of peptides which include potential receptor/channel effectors, and those peptides of the library which either agonize or antagonize the receptor or channel function can be selected and identified by sequence.

A “control cell” may be derived from the same cells from which the recombinant cell was prepared but which had not been modified by introduction of heterologous DNA, encoding the target receptor or which has not been contacted with a sub-threshold amount of a known agonist. Alternatively, it may be a cell in which the specific receptors are dysfunctional. Any statistically or otherwise significant difference in the amount of transcription indicates that the test modulating moiety has in some manner altered the activity of the specific receptor.

Host Cells:

Any transfectable cell that can express the desired cell surface protein in a manner such the protein functions to intracellularly transduce an extracellular signal may be used. The cells may be selected such that they endogenously express the target receptor protein or may be genetically engineered to do so.

Suitable host cells for generating the subject assay include prokaryotes, yeast, or higher eukaryotic cells, especially mammalian cells. Prokaryotes include gram negative or gram positive organisms. Examples of suitable mammalian host cell lines include the HEK 293, COS-7 line of monkey kidney cells (ATCC CRL 1651) (Gluzman (1981) Cell 23:175) CV-1 cells (ATCC CCL 70), L cells, C127, 3T3, Chinese hamster ovary (CHO), HeLa and BHK cell lines.

If yeast cells are used, the yeast may be of any species which are cultivable and in which an exogenous receptor can be made to engage the appropriate signal transduction machinery of the host cell. Suitable species include Kluyverei lactis, Schizosaccharomyces pombe, and Ustilaqo maydis; Saccharomyces cerevisiae is preferred. Other yeast which can be used in practicing the present invention are Neurospora crassa, Aspergillus niger, Aspergillus nidulans, Pichia pastoris, Candida tropicalis, and Hansenula polymorpha. The term “yeast”, as used herein, includes not only yeast in a strictly taxonomic sense, i.e., unicellular organisms, but also yeast-like multicellular fungi or filamentous fungi.

The choice of appropriate host cell will also be influenced by the choice of detection signal. For instance, second messenger generation can be measured directly in the detection step, such as mobilization of intracellular calcium or phospholipid metabolism are quantitated. Accordingly, it will be understood that to achieve selection or screening, the host cell must have an appropriate phenotype. For example, introducing a pheromone-responsive chimeric HIS3 gene into a yeast that has a wild-type HIS3 gene would frustrate genetic selection. Thus, to achieve nutritional selection, an auxotrophic strain is wanted.

In other embodiments, reporter constructs, as described below, can provide a selectable or screenable trait upon transcriptional activation (or inactivation) in response to a signal transduction pathway coupled to the target receptor. The reporter gene may be an unmodified gene already in the host cell pathway. It may be a host cell gene that has been operably linked to a “receptor-responsive” promoter. Alternatively, it may be a heterologous gene that has been so linked. Suitable genes and promoters are discussed below.

Expression Systems

Ligating a polynucleotide coding sequence into a gene construct, such as an expression vector, and transforming or transfecting into hosts, either eukaryotic (yeast, avian, insect or mammalian) or prokaryotic (bacterial cells), are standard procedures used in producing other well-known proteins, including sequences encoding exogenous receptor proteins (see, e.g., Sambrook et al. (1989) Molecular Cloning: A Laboratory Manual, Second Edition, Cold Spring Harbor Laboratory Press). Suitable means for introducing (transducing) expression vectors containing invention nucleic acid constructs into host cells to produce recombinant cells (i.e., cells containing recombinant heterologous nucleic acid) are also well-known in the art (see, for review, Friedmann, 1989, Science, 244:1275-1281; Mulligan, 1993, Science, 260:926-932, each of which are incorporated herein by reference in their entirety). Similar procedures, or modifications thereof, can be employed to prepare recombinant cells for use in the methods of the invention. Exemplary methods of transduction include, e.g., infection employing viral vectors (see, e.g., U.S. Pat. Nos. 4,405,712 and 4,650,764), calcium phosphate transfection (U.S. Pat. Nos. 4,399,216 and 4,634,665), dextran sulfate transfection, electroporation, lipofection (see, e.g., U.S. Pat. Nos. 4,394,448 and 4,619,794), cytofection, particle bead bombardment, and the like. The heterologous nucleic acid can optionally include sequences which allow for its extrachromosomal (i.e., episomal) maintenance, or the heterologous nucleic acid can be donor nucleic acid that integrates into the genome of the host. Recombinant cells can then be cultured under conditions whereby a target protein encoded by the DNA is (are) expressed. Preferred cells include mammalian cells (e.g., HEK 293, CHO and Ltk-cells), yeast cells (e.g., methylotrophic yeast cells, such as Pichia pastoris), bacterial cells (e.g., Escherichia coli), and the like.

As used herein, the term “vector” means an expression construct, e.g., nucleic acid construct wherein a DNA of interest operably linked to a suitable control sequence capable of effecting the expression of the DNA in a suitable host. Such control sequences include a promoter to effect transcription, an optional operator sequence to control such transcription, a sequence encoding suitable mRNA ribosome binding sites, and sequences which control the termination of transcription and translation. In the present specification, “plasmid” and “vector” are sometimes used interchangeably, as the plasmid is the most commonly used form of vector at present. However, the invention is intended to include such other forms of expression vectors which serve equivalent functions and which become known in the art subsequently hereto.

Expression cassette: is conventional and refers to a combination of regulatory elements that are required by the host for the correct transcription and translation (expression) of the genetic information contained in the expression cassette. These regulatory elements comprise a suitable (i.e., functional in the selected host) transcription promoter and a suitable transcription termination sequence.

A “promoter” is defined as an array of nucleic acid control sequences that direct transcription of a nucleic acid. As used herein, a promoter includes necessary nucleic acid sequences near the start site of transcription, such as, in the case of a polymerase II type promoter, a TATA element. A promoter also optionally includes distal enhancer or repressor elements, which can be located as much as several thousand base pairs from the start site of transcription. A “constitutive” promoter is a promoter that is active under most environmental and developmental conditions. An “inducible” promoter is a promoter that is active under environmental or developmental regulation. The term “operably linked” refers to a functional linkage between a nucleic acid expression control sequence (such as a promoter, or array of transcription factor binding sites) and a second nucleic acid sequence, wherein the expression control sequence directs transcription of the nucleic acid corresponding to the second sequence.

The terms “operably associated” and “operably linked” refer to functionally related but heterologous nucleic acid sequences. A promoter is operably associated or operably linked with a coding sequence if the promoter controls the translation or expression of the encoded polypeptide. While operably associated or operably linked nucleic acid sequences can be contiguous and in the same reading frame, certain genetic elements, e.g., repressor genes, are not contiguously linked to the sequence encoding the polypeptide but still bind to operator sequences that control expression of the polypeptide.

“Expression vector” includes vectors which are capable of expressing DNA sequences where such sequences are operably linked to other sequences capable of effecting their expression. It is implied, although not always explicitly stated, that these expression vectors must be replicable in the host organisms either as episomes or as an integral part of the chromosomal DNA. Clearly a lack of replicability would render them effectively inoperable. In sum, “expression vector” is given a functional definition, and any DNA sequence which is capable of effecting expression of a specified DNA code disposed therein is included in this term as it is applied to the specified sequence. In general, expression vectors of utility in recombinant DNA techniques are often in the form of “plasmids” which refer to circular double stranded DNA loops that, in their vector form are not bound to the chromosome.

Suitable expression vectors are well-known in the art, and include vectors capable of expressing DNA operatively linked to a regulatory sequence, such as a promoter region that is capable of regulating expression of such DNA. Thus, an expression vector refers to a recombinant DNA or RNA construct, such as a plasmid, a phage, recombinant virus or other vector that, upon introduction into an appropriate host cell, results in expression of the inserted DNA. Appropriate expression vectors are well known to those of skill in the art and include those that are replicable in eukaryotic cells and/or prokaryotic cells and those that remain episomal or those which integrate into the host cell genome.

As used herein, the term “expression” refers to any number of steps comprising the process by which nucleic acid molecules are transcribed into RNA, and (optionally) translated into peptides, polypeptides, or proteins. If the polynucleic acid is derived from genomic DNA, expression may, if an appropriate eukaryotic host cell or organism is selected, include splicing of the RNA.

The term “control sequences” refers to DNA sequences necessary for the expression of an operably linked coding sequence in a particular host organism. The control sequences that are suitable for prokaryotes, for example, include a promoter, optionally an operator sequence, and a ribosome binding site. Eukaryotic cells are known to utilize promoters, polyadenylation signals, and enhancers.

Appropriate cloning and expression vectors for use with bacterial, fungal, yeast, and mammalian cellular hosts are known in the art, and are described in, for example, Powels et al. (Cloning Vectors: A Laboratory Manual, Elsevier, New York, 1985). Mammalian expression vectors may comprise non-transcribed elements such as an origin of replication, a suitable promoter and enhancer linked to the gene to be expressed, and other 5′ or 3′ flanking nontranscribed sequences, and 5′ or 3′ nontranslated sequences, such as necessary ribosome binding sites, a poly-adenylation site, splice donor and acceptor sites, and transcriptional termination sequences.

Exemplary expression vectors for transformation of E. coli prokaryotic cells include the pET expression vectors (Novagen, Madison, Wis., see U.S. Pat. No. 4,952,496), e.g., pETlla, which contains the T7 promoter, T7 terminator, the inducible E. coli lac operator, and the lac repressor gene; and pET 12a-c, which contains the T7 promoter, T7 terminator, and the E. coli ompT secretion signal. Another such vector is the pIN-IIIompA2 (see Duffaud et al., Meth. in Enzymology, 153:492-507, 1987), which contains the 1 pp promoter, the lacUV5 promoter operator, the ompA secretion signal, and the lac repressor gene.

Exemplary eukaryotic expression vectors include eukaryotic cassettes, such as the pSV-2 gpt system (Mulligan et al., 1979, Nature, 277:108-114); the Okayama-Berg system (Mol. Cell Biol., 2:161-170), and the expression cloning vector described by Genetics Institute (1985, Science, 228:810-815). Each of these plasmid vectors is capable of promoting expression of the invention chimeric protein of interest.

Generally, recombinant expression vectors will include origins of replication and selectable markers permitting transfection of the host cell, e.g., the ampicillin resistance gene of E. coli and S. cerevisiae TRP1 gene, and a promoter derived from a highly-expressed gene to direct transcription of a downstream structural sequence. Such promoters can be derived from operons encoding glycolytic enzymes such as 3-phosphoglycerate kinase (PGK), α-factor, acid phosphatase, or heat shock proteins, among others. The heterologous structural sequence is assembled in appropriate phase with translation initiation and termination sequences. Optionally, the heterologous sequence can encode a fusion protein including an N-terminal identification peptide imparting desired characteristics, e.g., stabilization or simplified purification of expressed recombinant product.

The transcriptional and translational control sequences in expression vectors to be used in transforming mammalian cells may be provided by viral sources. For example, commonly used promoters and enhancers are derived from Polyoma, Adenovirus 2, Simian Virus 40 (SV40), and human cytomegalovirus. DNA sequences derived from the SV40 viral genome, for example, SV40 origin, early and late promoter, enhancer, splice, and polyadenylation sites may be used to provide the other genetic elements required for expression of a heterologous DNA sequence. The early and late promoters are particularly useful because both are obtained easily from the virus as a fragment which also contains the SV40 viral origin of replication (Fiers et al. (1978) Nature 273:111) Smaller or larger SV40 fragments may also be used, provided the approximately 250 bp sequence extending from the Hind III site toward the Bgl I site located in the viral origin of replication is included. Exemplary vectors can be constructed as disclosed by Okayama and Berg (1983, Mol. Cell Biol. 3:280). Other expression vectors for use in mammalian host cells are derived from retroviruses.

The use of viral transfection can also provide stably integrated copies of the expression construct. In particular, the use of retroviral, adenoviral or adeno-associated viral vectors is contemplated as a means for providing a stably transfected cell line which co-expresses an exogenous target cell surface receptor and transporter protein. In other embodiments, the recombinant cell may also express a polypeptide library whose actions on the target cell receptor are being investigated.

Particularly preferred vectors contain regulatory elements that can be linked to the target sequence for transfection of mammalian cells, and include cytomegalovirus (CMV) promoter-based vectors such as pcDNA1 (Invitrogen, San Diego, Calif.), MMTV promoter-based vectors such as pMAMNeo (Clontech, Palo Alto, Calif.), pIRES puro or pIRESneo (Clontech, Palo Alto) and pMSG (Pharmacia, Piscataway, N.J.), and SV40 promoter-based vectors such as pSVO (Clontech, Palo Alto, Calif.).

As noted, supra, the expression of the target heterologous nucleic acids in mammalian cells is preferred. Thus, for expression in mammalian cells, mammalian expression vectors will be required. Examples of mammalian expression vectors include pCDM8 (Seed, B. Nature 329:840(1987)) and pMT2PC (Kaufman et al., EMBO J 6:187-195 (1987)). Other preferred mammalian expression vectors that contain both prokaryotic sequences, to facilitate the propagation of the vector in bacteria, and one or more eukaryotic transcription units for expressing the target sequence in eukaryotic host cells. Exemplary vectors that can be readily adapted for use in the subject method include the pcDNAI/amp, pcDNAI/neo, pRc/CMV, pSV2gpt, pSV2neo, pSV2-dhfr, pTk2, pRSVneo, pMSG, pSVT7, pko-neo and pHyg derived vectors. Some of these vectors may be modified with sequences from bacterial plasmids, such as pBR322, to facilitate replication and drug resistance selection in both prokaryotic and eukaryotic cells.

On the other hand, derivatives of viruses, such as the bovine papillomavirus (BPV-1), or Epstein-Barr virus (pHEBo, pREP-derived and p205) and the like, may also find use in the claimed method(s) of the invention. The various methods employed in the preparation of the plasmids are well known in the art.

In some instances, it may be desirable to derive the host cell using insect cells. In such embodiments, recombinant polypeptides can be expressed by the use of a baculovirus expression system. Examples of such baculovirus expression systems include pVL-derived vectors (such as pVL1392, pVL1393 and pVL941), pAcUW-derived vectors (such as pAcUWI), and pBlueBac-derived vectors (such as the B-gal containing pBlueBac III).

G Protein-Coupled Receptors.

One family of signal transduction cascades found in eukaryotic cells utilizes heterotrimeric “G proteins.” Many different G proteins are known to interact with receptors. G protein signaling systems include three components: the receptor itself, a GTP-binding protein (G protein), and an intracellular target protein, wherein the cell membrane acts as a switchboard. Thus, messages arriving through different receptors may produce a single effect if the receptors act on the same type of G protein. On the other hand, signals activating a single receptor can produce more than one effect if the receptor acts on different kinds of G proteins, or if the G proteins can act on different effectors.

The phrase “functional effects” in the context of assays for testing compounds that modulate GPCR-mediated signal transduction includes the determination of any parameter that is indirectly or directly under the influence of a GPCR, e.g., a functional, physical, or chemical effect. It includes ligand binding, changes in ion flux, membrane potential, current flow, transcription, G-protein binding, gene amplification, expression in cancer cells, GPCR phosphorylation or dephosphorylation, signal transduction, receptor-ligand interactions, second messenger concentrations (e.g., cAMP, cGMP, IP₃, or intracellular Ca²⁺), in vitro, in vivo, and ex vivo and also includes other physiologic effects such increases or decreases of neurotransmitter or hormone release.

By “determining the functional effect” is meant assays for a compound that increases or decreases a parameter that is indirectly or directly under the influence of a GPCR, e.g., functional, physical and chemical effects. Such functional effects can be measured by any means known to those skilled in the art, e.g., changes in spectroscopic characteristics (e.g., fluorescence, absorbance, refractive index), hydrodynamic (e.g., shape), chromatographic, or solubility properties, patch clamping, voltage-sensitive dyes, whole cell currents, radioisotope efflux, inducible markers, transcriptional activation of GPCRs; ligand binding assays; voltage, membrane potential and conductance changes; ion flux assays; changes in intracellular second messengers such as cAMP and inositol triphosphate (IP3); changes in intracellular calcium levels; neurotransmitter release, and the like.

“Inhibitors,” “activators,” and “modulators” of GPCRs refer to inhibitory, activating, or modulating molecules identified using in vitro and in vivo assays for signal transduction, e.g., ligands, agonists, antagonists, and their homologs and mimetics. Such modulating molecules, also referred to herein as compounds, include polypeptides, antibodies, amino acids, nucleotides, lipids, carbohydrates, or any organic or inorganic molecule. Inhibitors are compounds that, e.g., bind to, partially or totally block stimulation, decrease, prevent, delay activation, inactivate, desensitize, or down regulate signal transduction, e.g., antagonists. Activators are compounds that, e.g., bind to, stimulate, increase, open, activate, facilitate, enhance activation, sensitize or up regulate signal transduction, e.g., agonists. Modulators include compounds that, e.g., alter the interaction of a polypeptide with: extracellular proteins that bind activators or inhibitors; G-proteins; G-protein α, β, and γ subunits; and kinases. Modulators also include genetically modified versions of GPCRs, e.g., with altered activity, as well as naturally occurring and synthetic ligands, antagonists, agonists, antibodies, small chemical molecules and the like. Such assays for inhibitors and activators include, e.g., expressing GPCRs in vitro, in cells, or cell membranes, applying putative modulator compounds, and then determining the functional effects on signal transduction, as described above.

The “exogenous target cell surface receptors” of the present invention may be any G protein-coupled receptor which is exogenous to the cell which is to be genetically engineered for the purpose of the present invention. This receptor may be a plant or animal cell receptor. Screening for binding to plant cell receptors may be useful in the development of, e.g., herbicides. In the case of an animal receptor, it may be of invertebrate or vertebrate origin. If an invertebrate receptor, a mammalian receptor is preferred, more preferably a human receptor protein, and would facilitate development of therapeutics for treating mammalian metabotropic glutamate receptor protein mediated disorders.

Heterologous Polypeptide refers to a linear series of amino acid residues connected one to the other by peptide bonds between the α-amino and carboxy groups of adjacent residues originating from a species other than the plant host system within which said linear series is produced. “Polypeptide” also encompasses a sequence of amino acids, peptides, fragments of polypeptides, proteins, globular proteins, glycoproteins, and fragments of these.

Known ligands for G protein coupled receptors include: purines and nucleotides, such as adenosine, cAMP, ATP, UTP, glutamate, melatonin and the like; biogenic amines (and related natural ligands), such as 5-hydroxytryptamine, acetylcholine, dopamine, adrenaline, adrenaline, adrenaline., histamine, noradrenaline, noradrenaline, noradrenaline, tyramine/octopamine and other related compounds; peptides such as adrenocorticotrophic hormone (acth), melanocyte stimulating hormone (msh), melanocortins, neurotensin (nt), bombesin and related peptides, endothelins, cholecystokinin, gastrin, neurokinin b (nk3), invertebrate tachykinin-like peptides, substance k (nk2), substance p (nk1), neuropeptide y (npy), thyrotropin releasing-factor (trf), bradykinin, angiotensin ii, β-endorphin, c5a anaphalatoxin, calcitonin, chemokines (also called intercrines), corticotrophic releasing factor (crf), dynorphin, endorphin, fmlp and other formylated peptides, follitropin (fsh), fungal mating pheremones, galanin, gastric inhibitory polypeptide receptor (gip), glucagon-like peptides (glps), glucagon, gonadotropin releasing hormone (gnrh), growth hormone releasing hormone(ghrh), insect diuretic hormone, interleukin-8, leutropin (1h/hcg), met-enkephalin, opioid peptides, oxytocin, parathyroid hormone (pth) and pthrp, pituitary adenylyl cyclase activiating peptide (pacap), secretin, somatostatin, thrombin, thyrotropin (tsh), vasoactive intestinal peptide (vip), vasopressin, vasotocin; eicosanoids such as ip-prostacyclin, pg-prostaglandins, tx-thromboxanes; retinal based compounds such as vertebrate 11-cis retinal, invertebrate 11-cis retinal and other related compounds; lipids and lipid-based compounds such as cannabinoids, anandamide, lysophosphatidic acid, platelet activating factor, leukotrienes and the like; excitatory amino acids and ions such as calcium ions and glutamate.

Suitable examples of G-protein coupled receptors include, but are not limited to, metabotropic glutamate receptors, although other receptor may work just as well. The term “receptor,” as used herein, encompasses both naturally occurring and mutant receptors.

The discovery that glutamate is a ligand of the human metabotropic glutamate receptor protein (hmGluR) receptor permits screening assays to identify agonists, antagonists and inverse agonists of receptor activity.

Screening Procedures

Assays for the Identification of Agents that Modulate Target Cell Surface Receptor(s)

Agents that modulate the activity of human metabotropic glutamate receptor can be identified in a number of ways that take advantage of the interaction of the receptor with glutamate. For example, the ability to reconstitute human metabotropic glutamate receptor/glutamate binding either in vitro, on cultured cells or in vivo provides a target for the identification of agents that disrupt that binding. Assays based on disruption of binding can identify agents, such as small organic molecules, from libraries or collections of such molecules. Alternatively, such assays can identify agents in samples or extracts from natural sources, e.g., plant, fungal or bacterial extracts or even in human tissue samples (e.g., tumor tissue). In one aspect, the extracts can be made from cells expressing a library of variant nucleic acids, peptides or polypeptides, including, for example, variants of glutamate itself. Modulators of human metabotropic glutamate receptor/glutamate binding can then be screened using a binding assay or a functional assay that measures downstream signaling through the receptor. Both binding assays and functional assays are validated using glutamate.

Another approach that uses the human metabotropic glutamate receptor/glutamate interaction more directly to identify agents that modulate human metabotropic glutamate receptor function measures changes in human metabotropic glutamate receptor downstream signaling induced by candidate agents or candidate modulators. These functional assays can be performed in isolated cell membrane fractions or on cells expressing the receptor on their surfaces.

Thus, metabotropic glutamate receptor antagonists may be identified by their ability to inhibit or reduce stimulation of cAMP production, relative to the cAMP production in the presence of native metabotropic glutamate receptor and a known agonist, as determined in an adenylate cyclase assay. Adenylate cyclase assays are described, for example, by Lin et al. (Biochemistry 14:1559-1563, 1975; which is incorporated herein by reference in its entirety). Biological responses via the inositol triphosphate pathway may be assessed by measuring inositol phosphate metabolism as generally described in Subers and Nathanson (J. Mol. Cell. Cardiol. 20:131-140, 1988; which is incorporated herein by reference in its entirety) or Pittner and Fain (ibid.; which is incorporated herein by reference in its entirety) or by measuring the intracellular calcium concentration as generally described by Grynkiewicz et al. (J. Biol. Chem. 260:3440-3450, 1985; which is incorporated herein by reference in its entirety).

The discovery that glutamate is a ligand of the human metabotropic glutamate receptor permits screening assays to identify agonists, antagonists, postive and negative allosteric modulators and inverse agonists of receptor activity. The screening assays will have two general approaches.

1) Ligand binding assays, in which cells co-expressing a human metabotropic glutamate receptor and a glutamate transport protein, membrane extracts from such cells, or immobilized lipid membranes comprising human metabotropic glutamate receptor are exposed to a labeled glutamate and candidate compound. Following incubation, the reaction mixture is measured for specific binding of the labeled glutamate to the human metabotropic glutamate receptor. Compounds that interfere with or displace labeled glutamate can be agonists, antagonists or inverse agonists of human metabotropic glutamate receptor activity. Functional analysis can be performed on positive compounds to determine which of these categories they fit.

2) Functional assays, in which a signaling activity of human metabotropic glutamate receptor is measured.

a) For agonist screening, cells co-expressing a human metabotropic glutamate receptor and a glutamate transport protein or membranes prepared from them are incubated with candidate compound, and a signaling activity of human metabotropic glutamate receptor is measured. The assays are validated using glutamate as agonist, and the activity induced by compounds that modulate receptor activity is compared to that induced by glutamate. An agonist or partial agonist will have a maximal biological activity corresponding to at least 10% of the maximal activity of wild type human glutamate when the agonist or partial agonist is present at 10 μor less, and preferably will have 50%, 75%, 100% or more, including 2-fold, 5-fold, 10-fold or more activity than wild-type human glutamate.

b) For antagonist or inverse agonist screening, cells co-expressing a human metabotropic glutamate receptor and a glutamate transport protein or membranes isolated from them are assayed for signaling activity in the presence of glutamate with or without a candidate compound. Antagonists or inverse agonists will reduce the level of glutamate-stimulated receptor activity by at least 10%, relative to reactions lacking the antagonist or inverse agonist.

As understood by those of skill in the art, assay methods for identifying compounds that modulate human metabotropic glutamate receptor activity (e.g., agonists and antagonists) generally require comparison to a control. One type of a “control” cell or “control” culture is a cell or culture that is treated substantially the same as the cell or culture exposed to the test compound, except the control culture is not exposed to test compound. Another type of “control” cell or “control” culture may be a cell or a culture of cells which are identical to the transfected cells, except the cells employed for the control culture do not express the recombinant human metabotropic glutamate receptor subtype(s) expressed in the transfected cells. In this situation, the response of test cell to test compound is compared to the response (or lack of response) of receptor-negative (control) cell to test compound, when cells or cultures of each type of cell are exposed to substantially the same reaction conditions in the presence of compound being assayed.

c) For inverse agonist screening, cells expressing constitutive human metabotropic glutamate receptor activity or membranes isolated from them are used in a functional assay that measures an activity of the receptor in the presence and absence of a candidate compound. Inverse agonists are those compounds that reduce the constitutive activity of the receptor by at least 10%. Overexpression of human metabotropic glutamate receptor (i.e., expression of 5-fold or higher excess of human metabotropic glutamate receptor polypeptide relative to the level naturally expressed in macro phages in vivo) may lead to constitutive activation.

Ligand Binding And Displacement Assays:

One can use human metabotropic glutamate receptor polypeptides expressed on a cell, or isolated membranes containing receptor polypeptides, along with glutamate in order to screen for compounds that inhibit the binding of glutamate to human metabotropic glutamate receptor. When identified in an assay that measures binding or glutamate displacement alone, compounds will have to be subjected to functional testing to determine whether they act as agonists, antagonists or inverse agonists.

For displacement experiments, cells expressing a human metabotropic glutamate receptor polypeptide (generally 25,000 cells per assay or 1 to 100 μg of membrane extracts) are incubated in binding buffer (e.g., 50 mM Hepes pH 7.4; 1 mM CaCl⁺⁺; 0.5% Bovine Serum Albumin (BSA) Fatty Acid-Free; and 0 5 mM MgCl 2) for 1.5 hrs (at, for example, 27° C.) with labeled glutamate in the presence or absence of increasing concentrations of a candidate modulator. To validate and calibrate the assay, control competition reactions using increasing concentrations of unlabeled glutamate can be performed. After incubation, cells are washed extensively, and bound, labeled glutamate is measured as appropriate for the given label (e.g., scintillation counting, enzyme assay, fluorescence, etc.). A decrease of at least 10% in the amount of labeled glutamate bound in the presence of candidate modulator indicates displacement of binding by the candidate modulator. Candidate modulators are considered to bind specifically in this or other assays described herein if they displace 50% of labeled glutamate (sub-saturating glutamate dose) at a concentration of 10 μM or less (i.e., EC₅₀ is 10 μM or less).

Alternatively, binding or displacement of binding can be monitored by surface plasmon resonance (SPR). Surface plasma resonance assays can be used as a quantitative method to measure binding between two molecules by the change in mass near an immobilized sensor caused by the binding or loss of binding of glutamate from the aqueous phase to a human metabotropic glutamate receptor polypeptide immobilized in a membrane on the sensor. This change in mass is measured as resonance units versus time after injection or removal of the glutamate or candidate modulator and is measured using a Biacore Biosensor (Biacore AB). Human metabotropic glutamate receptor can be immobilized on a sensor chip (for example, research grade CM5 chip; Biacore AB) in a thin film lipid membrane according to methods described by Salamon et al. (Salamon et al., 1996, Biophys J. 71: 283-294; Salamon et al., 2001, Biophys. J. 80: 1557-1567; Salamon et al., 1999, Trends Biochem. Sci. 24: 213-219, each of which is incorporated herein by reference.). Sarrio et al. demonstrated that SPR can be used to detect ligand binding to the GPCR A(1) adenosine receptor immobilized in a lipid layer on the chip (Sarrio et al., 2000, Mol. Cell. Biol. 20: 5164-5174, incorporated herein by reference). Conditions for glutamate binding to human metabotropic glutamate receptor in an SPR assay can be fine-tuned by one of skill in the art using the conditions reported by Sarrio et al. as a starting point.

SPR can assay for modulators of binding in at least two ways. First, glutamate can be pre-bound to immobilized human metabotropic glutamate receptor polypeptide, followed by injection of candidate modulator at approximately 10 μl/min flow rate and a concentration ranging from 1 nM to 100 μM, preferably about 1 μM Displacement of the bound glutamate can be quantitated, permitting detection of modulator binding. Alternatively, the membrane-bound human metabotropic glutamate receptor polypeptide can be pre-incubated with candidate modulator and challenged with glutamate. A difference in glutamate binding to the human metabotropic glutamate receptor exposed to modulator relative to that on a chip not pre-exposed to modulator will demonstrate binding. In either assay, a decrease of 10% or more in the amount of glutamate bound is in the presence of candidate modulator, relative to the amount of glutamate bound in the absence of candidate modulator indicates that the candidate modulator inhibits the interaction of human metabotropic glutamate receptor and glutamate.

Any of the binding assays described can be used to determine the presence of an agent in a sample, e.g., a tissue sample, that binds to the human metabotropic glutamate receptor receptor molecule, or that affects the binding of glutamate to the receptor. To do so, human metabotropic glutamate receptor polypeptide is reacted with glutamate or another ligand in the presence or absence of the sample, and glutamate or ligand binding is measured as appropriate for the binding assay being used. A decrease of 10% or more in the binding of glutamate or other ligand indicates that the sample contains an agent that modulates glutamate or ligand binding to the receptor polypeptide.

The following is a description of procedures that can be used to obtain compounds modulating metabotropic glutamate receptor activity. Various screening procedures can be carried out to assess the ability of a compound to modulate activity of chimeric receptors of the invention by measuring its ability to have one or more activities of a metabotropic glutamate receptor modulating agent or a calcium receptor modulating agent. In cells expressing chimeric receptors of the invention, such activities include the effects on intracellular calcium, inositol phosphates and cyclic AMP.

Screening and Selection: Assays of Second Messenger Generation

GTPase/GTP Binding Assays: For GPCRs such as human metabotropic glutamate receptor, a measure of receptor activity is the binding of GTP by cell membranes containing receptors. In the method described by Traynor and Nahorski, 1995, Mol. Pharmacol. 47: 848-854, incorporated herein by reference, one essentially measures G-protein coupling to membranes by measuring the binding of labeled GTP. For GTP binding assays, membranes isolated from cells co-expressing the mGluR receptor and the transport protein are incubated in a buffer containing 20 mM HEPES, pH 7.4, 100 mM NaCl, and 10 mM MgCl2, 80 pM³⁵S-GTPγS and 3 μM GDP. The assay mixture is incubated for 60 minutes at 30° C., after which unbound labeled GTP is removed by filtration onto GF/B filters. Bound, labeled GTP is measured by liquid scintillation counting. In order to assay for modulation of glutamate-induced human metabotropic glutamate receptor activity, membranes prepared from cells co-expressing a human metabotropic glutamate receptor polypeptide and a glutamate transport protein (mGLAST)are mixed with glutamate, and the GTP binding assay is performed in the presence and absence of a candidate modulator of human metabotropic glutamate receptor activity. A decrease of 10% or more in labeled GTP binding as measured by scintillation counting in an assay of this kind containing candidate modulator, relative to an assay without the modulator, indicates that the candidate modulator inhibits human metabotropic glutamate receptor activity.

A similar GTP-binding assay can be performed without glutamate to identify compounds that act as agonists. In this case, glutamate-stimulated GTP binding is used as a standard. A compound is considered an agonist if it induces at least 50% of the level of GTP binding induced by full length wild-type glutamate when the compound is present at 1 μM or less, and preferably will induce a level the same as or higher than that induced by glutamate.

GTPase activity is measured by incubating the membranes containing a human metabotropic glutamate receptor polypeptide with γ 32P-GTP. Active GTPase will release the label as inorganic phosphate, which is detected by separation of free inorganic phosphate in a 5% suspension of activated charcoal in 20 mM H₃PO₄, followed by scintillation counting. Controls include assays using membranes isolated from cells not co-expressing a human metabotropic glutamate receptor and a glutamate transport protein (mock-transfected), in order to exclude possible non-specific effects of the candidate compound.

In order to assay for the effect of a candidate modulator on human metabotropic glutamate receptor-regulated GTPase activity, membrane samples are incubated with glutamate, with and without the modulator, followed by the GTPase assay. A change (increase or decrease) of 10% or more in the level of GTP binding or GTPase activity relative to samples without modulator is indicative of human metabotropic glutamate receptor modulation by a candidate modulator.

Cells may be screened for the presence of endogenous mammalian receptor using radioligand binding or functional assays (described in detail in the above or following experimental description, respectively). Cells with no or a low level of endogenous receptor present may be transfected with the mammalian receptor for use in the following functional assays.

A wide spectrum of assays can be employed to screen for the presence of receptor ligands. These range from traditional measurements of phosphatidyl inositol, cAMP, Ca²⁺ and K⁺, for example; to systems measuring these same second messengers but which have been modified or adapted to be higher throughput, more generic, and more sensitive.

Downstream Pathway Activation Assays:

Measuring [Ca²⁺]intracellular with fura-2 provides a very rapid means of screening new organic molecules for activity. In a single afternoon, 10-15 compounds (or molecule types) can be examined and their ability to mobilize or inhibit mobilization of intracellular Ca²⁺ can be assessed by a single experiment. The sensitivity of observed increases in [Ca²⁺]intracellular to depression by PMA can also be assessed.

For example, recombinant cells co-expressing one or more metabotropic glutamate receptors and a glutamate transport protein are loaded with fura-2 are initially suspended in buffer containing 0.5 mM CaCl₂. A test substance is added to the cuvette in a small volume (5-15 μl) and changes in the fluorescence signal are measured. Cumulative increases in the concentration of the test substance are made in the cuvette until some predetermined concentration is achieved or no further changes in fluorescence are noted. If no changes in fluorescence are noted, the molecule is considered inactive and no further testing is performed.

In the initial studies, molecules may be tested at concentrations as high as 5 or 10 mM. As more potent molecules became known, the ceiling concentration was lowered. For example, newer molecules are tested at concentrations no greater than 500 μM. If no changes in fluorescence are noted at this concentration, the molecule can be considered inactive.

Molecules causing increases in Ca²⁺ᵢ are subjected to additional testing. Two characteristics of a molecule which can be considered in screening for a positive modulating agent of a chimeric receptor of the invention are the mobilization of intracellular calciumand sensitivity to PKC activators.

A single preparation of cells can provide data on intracellular calcium, cyclic AMP levels, IP₃ and other intracellular messengers. A typical procedure is to load cells with fura-2 and then divide the cell suspension in two; most of the cells are used for measurement of [Ca²⁺]ᵢ and the remainder are incubated with molecules to assess their effects on cyclic AMP.

Measurements of inositol phosphates are a time-consuming aspect of the screening. However, ion-exchange columns eluted with chloride (rather than formate) provide a very rapid means of screening for IP₃ formation, since rotary evaporation (which takes around 30 hours) is not required. This method allows processing of nearly 100 samples in a single afternoon by a single experimenter. Those molecules that prove interesting, as assessed by measurements of [Ca²⁺]ᵢ cyclic AMP, and IP₃ can be subjected to a more rigorous analysis by examining formation of various inositol phosphates and assessing their isomeric form by HPLC.

The following is illustrative of methods useful in these screening procedures.

a. Cyclic AMP (cAMP) Formation Assay

The receptor-mediated inhibition of cyclic AMP (cAMP) formation may be assayed in transfected cells expressing the mammalian receptors. Cells are plated in 96-well plates and incubated in Dulbecco's phosphate buffered saline (PBS) supplemented with 10 mM HEPES, 5 mM theophylline, 2 μg/ml aprotinin, 0.5 mg/ml leupeptin, and 10 μg/ml phosphoramidon for 20 min at 37° C., in 5% CO₂. Test compounds are added and incubated for an additional 10 min at 37° C. The medium is then aspirated and the reaction stopped by the addition of 100 mM HCl. The plates are stored at 4° C. for 15 min, and the cAMP content in the stopping solution measured by radioimmunoassay. Radioactivity may be quantified using a γ counter equipped with data reduction software.

Intracellular or extracellular cAMP is measured using a cAMP radioimmunoassay (RIA) or cAMP binding protein according to methods widely known in the art. For example, Horton & Baxendale, 1995, Methods Mol. Biol. 41: 91-105, which is incorporated herein by reference, describes a RIA for cAMP. A number of kits for the measurement of cAMP are commercially available, such as the High Efficiency Fluorescence Polarization-based homogeneous assay marketed by LJL Biosystems and NEN Life Science Products. Control reactions should be performed using extracts of mock-transfected cells to exclude possible non-specific effects of some candidate modulators.

The level of cAMP is “changed” if the level of cAMP detected in cells, expressing a human metabotropic glutamate receptor polypeptide and treated with a candidate modulator of human metabotropic glutamate receptor activity (or in extracts of such cells), using the RIA-based assay of Horton & Baxendale, 1995, supra, increases or decreases by at least 10% relative to the cAMP level in similar cells not treated with the candidate modulator.

b. Arachidonic Acid Release Assay

Cells stably transfected with the mammalian receptor are seeded into 96 well plates and grown for 3 days in HAM's F-12 with supplements. ³H-arachidonic acid (specific activity=0.75 μCi/ml) is delivered as a 100 μL aliquot to each well and samples were incubated at 37° C., 5% CO₂ for 18 hours. The labeled cells are washed three times with 200 μL HAM's F-12. The wells are then filled with medium (200 μL) and the assay is initiated with the addition of peptides or buffer (22 μL). Cells are incubated for 30 min at 37° C., 5% CO₂. Supernatants are transferred to a microtiter plate and evaporated to dryness at 75° C. in a vacuum oven. Samples are then dissolved and resuspended in 25 μL distilled water. Scintillant (300 μL) is added to each well and samples are counted for ³H in a Trilux plate reader. Data are analyzed using nonlinear regression and statistical techniques available in the GraphPAD Prism package (San Diego, Calif.).

c. Adenylate Cyclase Assay:

Assays for adenylate cyclase activity are described by Kenimer & Nirenberg, 1981, Mol. Pharmacol. 20: 585-591, incorporated herein by reference. That assay is a modification of the assay taught by Solomon et al., 1974, Anal. Biochem. 58: 541-548, also incorporated herein by reference. Briefly, 100 μl reactions contain 50 mM Tris-Hcl (pH 7.5), 5 mM MgCl₂, 20 mM creatine phosphate (disodium salt), 10 units (71 μg of protein) of creatine phosphokinase, 1 mM α-³²P-ATP (tetrasodium salt, 2 .mCi), 0.5 mM cyclic AMP, G-³H-labeled cyclic AMP (approximately 10,000 cpm), 0.5 mM Ro20-1724, 0.25% ethanol, and 50-200 μg of protein homogenate to be tested (i.e., homogenate from cells expressing or not expressing a human metabotropic glutamate receptor polypeptide, treated or not treated with glutamate with or without a candidatemodulator). Reaction mixtures are generally incubated at 37° C. for 6 minutes. Following incubation, reaction mixtures are deproteinized by the addition of 0.9 ml of cold 6% trichloroacetic acid. Tubes are centrifuged at 1800×g for 20 minutes and each supernatant solution is added to a Dowex AG50W-X4 column. The cAMP fraction from the column is eluted with 4 ml of 0.1 mM imidazole-HCl (pH 7.5) into a counting vial. Assays should be performed in triplicate. Control reactions should also be performed using protein homogenate from cells that do not express a human metabotropic glutamate receptor polypeptide.

According to the invention, adenylate cyclase activity is “changed” if it increases or decreases by 10% or more in a sample taken from cells treated with a candidate modulator of human metabotropic glutamate receptor activity, relative to a similar sample of cells not treated with the candidate modulator or relative to a sample of cells not expressing the human metabotropic glutamate receptor polypeptide (mock-transfected cells) but treated with the candidate modulator.

d. Phospholipid Breakdown, DAG Production and Inositol Triphosphate Levels:

Receptors that activate the breakdown of phospholipids can be monitored for changes due to the activity of known or suspected modulators of human metabotropic glutamate receptor by monitoring phospholipid breakdown, and the resulting production of second messengers DAG and/or inositol triphosphate (IP₃). Methods of measuring each of these are described in Phospholipid Signaling Protocols, edited by Ian M. Bird. Totowa, N.J., Humana Press, 1998, which is incorporated herein by reference. See also Rudolph et al., 1999, J. Biol. Chem. 274: 11824-11831, incorporated herein by reference, which also describes an assay for phosphatidylinositol breakdown. Assays should be performed using cells or extracts of cells co-expressing a human metabotropic glutamate receptor and a glutamate transport protein, treated or not treated with glutamate with or without a candidate modulator. Control reactions should be performed using mock-transfected cells, or extracts from them in order to exclude possible non-specific effects of some candidate modulators.

According to the invention, phosphatidylinositol breakdown, and diacylglycerol and/or inositol triphosphate levels are “changed” if they increase or decrease by at least 10% in a sample from cells expressing a human metabotropic glutamate receptor polypeptide and treated with a candidate modulator, relative to the level observed in a sample from cells expressing a human metabotropic glutamate receptor polypeptide that is not treated with the candidate modulator.

Metabotropic glutamate receptor-mediated activation of the inositol phosphate (IP) second messenger pathways can be assessed by radiometric measurement of IP products. In a 96 well microplate format assay, cells are plated at a density of 70,000 cells per well and allowed to incubate for 24 hours. The cells are then labeled with 0.5 μCi [³H]-myo-inositol overnight at 37° C., 5% CO₂. Immediately before the assay, the medium is removed and replaced with 90. μL of PBS containing 10 mM LiCl. The plates are then incubated for 15 min at 37° C., 5% CO₂. Following the incubation, the cells are challenged with agonist (10 μL/well; 10×concentration) for 30 min at 37° C., 5% CO₂. The challenge is terminated by the addition of 100 μL of 50% v/v trichloroacetic acid, followed by incubation at 4° C. for greater than 30 minutes. Total IPs are isolated from the lysate by ion exchange chromatography. Briefly, the lysed contents of the wells are transferred to a Multiscreen HV filter plate (Millipore) containing Dowex AG1-X8 (200-400 mesh, formate form). The filter plates are prepared adding 100 μL of Dowex AG1-X8 suspension (50% v/v, water: resin) to each well. The filter plates are placed on a vacuum manifold to wash or elute the resin bed. Each well is first washed 2 times with 200 μl of 5 mM myo-inositol. Total [³H]inositol phosphates are eluted with 75. μl of 1.2M ammonium formate/0.1M formic acid solution into 96-well plates. 200 μl of scintillation cocktail is added to each well, and the radioactivity is determined by liquid scintillation counting.

e. PKC Activation Assays:

Growth factor receptor tyrosine kinases tend to signal via a pathway involving activation of Protein Kinase C (PKC), which is a family of phospholipid- and calcium-activated protein kinases. PKC activation ultimately results in the transcription of an array of proto-oncogene transcription factor-encoding genes, including c-fos, c-myc and c-jun, proteases, protease inhibitors, including collagenase type I and plasminogen activator inhibitor, and adhesion molecules, including intracellular adhesion molecule I (ICAM I). Assays designed to detect increases in gene products induced by PKC can be used to monitor PKC activation and thereby receptor activity. In addition, the activity of receptors that signal via PKC can be monitored through the use of reporter gene constructs driven by the control sequences of genes activated by PKC activation. This type of reporter gene-based assay is discussed in more detail below.

For a more direct measure of PKC activity, the method of Kikkawa et al., 1982, J. Biol. Chem. 257: 13341, incorporated herein by reference, can be used. This assay measures phosphorylation of a PKC substrate peptide, which is subsequently separated by binding to phosphocellulose paper. This PKC assay system can be used to measure activity of purified kinase, or the activity in crude cellular extracts. Protein kinase C sample can be diluted in 20 mM HEPES/2 mM DTT immediately prior to assay.

The substrate for the assay is the peptide Ac-FKKSFKL-NH2, derived from the myristoylated alanine-rich protein kinase C substrate protein (MARCKS). The Kₘ of the enzyme for this peptide is approximately 50 μM. Other basic, protein kinase C-selective peptides known in the art can also be used, at a concentration of at least 2-3 times their Kₘ Cofactors required for the assay include calcium, magnesium, ATP, phosphatidylserine and diacylglycerol. Depending upon the intent of the user, the assay can be performed to determine the amount of PKC present (activating conditions) or the amount of active PCK present (non-activating conditions). For most purposes according to the invention, non-activating conditions will be used, such that the PKC that is active in the sample when it is isolated is measured, rather than measuring the PKC that can be activated. For non-activating conditions, calcium is omitted in the assay in favor of EGTA.

The assay is performed in a mixture containing 20 mM HEPES, pH 7.4, 1-2 mM DTT, 5 mM MgCl₂, 100 . μM ATP, .about.1 μCi γ-³²P-ATP, 100 μg/ml peptide substrate (.about.100 μM), 140 μM/3.8 μM phosphatidylserine/diacylglycerol membranes, and 100 μM calcium (or 500 μM EGTA). 48 μl of sample, diluted in 20 mM HEPES, pH 7.4, 2 mM DTT is used in a final reaction volume of 80 μl. Reactions are performed at 30° C. for 5-10 minutes, followed by addition of 25 μl of 100 mM ATP, 100 mM EDTA, pH 8.0, which stops the reactions.

After the reaction is stopped, a portion (85 .μl) of each reaction is spotted onto a Whatman P81 cellulose phosphate filter, followed by washes: four times 500 ml in 0.4% phosphoric acid, (5-10 min per wash); and a final wash in 500 ml 95% EtOH, for 2-5 min. Bound radioactivity is measured by scintillation counting. Specific activity (cpm/nmol) of the labeled ATP is determined by spotting a sample of the reaction onto P81 paper and counting without washing. Units of PKC activity, defined as nmol phosphate transferred per min, are calculated as follows:
The activity, in UNITS (nmol/min) is: 1 The activity, in UNITS (n mol/min) is:=(cpm on paper)×(105 1 total/85 1 spotted) (assay time, min) (specific activity of ATP cpm/n mol).

An alternative assay can be performed using a Protein Kinase C Assay Kit sold by PanVera (Cat. #P2747).

Assays are performed on extracts from cells expressing a human metabotropic glutamate receptor polypeptide, treated or not treated with glutamate with or without a candidate modulator. Control reactions should be performed using mock-transfected cells, or extracts from them in order to exclude possible non-specific effects of some candidate modulators.

According to the invention, PKC activity is “changed” by a candidate modulator when the units of PKC measured by either assay described above increase or decrease by at least 10%, in extracts from cells co-expressing a human metabotropic glutamate receptor and a glutamate transport protein and treated with a candidate modulator, relative to a reaction performed on a similar sample from cells not treated with a candidate modulator.

f. GTPγS Functional Assay

Membranes from cells expressing the receptor are suspended in assay buffer (e.g., 50 mM Tris, 100 mM NaCl, 5 mM MgCl₂, 10 μM GDP, pH 7.4) with or without protease inhibitors (e.g., 0.1% bacitracin). Membranes are incubated on ice for 20 minutes, transferred to a 96-well Millipore microtiter GF/C filter plate and mixed with GTPγ³⁵S (e.g., 250,000 cpm/sample, specific activity .about.1000 Ci/mmol) plus or minus unlabeled GTPγS (final concentration=100 μM). Final membrane protein concentration.apprxeq.90 μg/ml. Samples are incubated in the presence or absence of test compounds for 30 min. at room temperature, then filtered on a Millipore vacuum manifold and washed three times with cold (4.degree. C.) assay buffer. Samples collected in the filter plate are treated with scintillant and counted for ³⁵S in a Trilux (Wallac) liquid scintillation counter. It is expected that optimal results are obtained when the receptor membrane preparation is derived from an appropriately engineered heterologous expression system, i.e., an expression system resulting in high levels of expression of the receptor and/or expressing G-proteins having high turnover rates (for the exchange of GDP for GTP). GTPγS assays are well-known to those skilled in the art, and it is contemplated that variations on the method described above, such as are described by Tian et al. (1994) or Lazareno and Birdsall (1993), may be used.

g. Intracellular Calcium Mobilization Assay

Intracellular calcium concentration (Ca²⁺i) acts as a modulator of many important physiological responses and pathophysiological conditions such as excitotoxic brain damage (B. K. Siesjo, Magnesium 8, 223 (1989)). In most of these events extracellular signals are received through receptors and converted to changes in >Ca2+ᵢ. This leads to less well characterized >Ca2+ᵢ sensitive changes inside the cell, possibly including modulation of >Ca²⁺ sensitive kinases, proteases and transcription factors (M. L. Villereal and H. C. Palfrey, Annu. Rev. Nutr. 9, 347 (1989)). Measurement of >Ca²⁺ is essential in understanding such modulation. Modified methods for detecting receptor-mediated signal transduction exist and one of skill in the art will recognize suitable methods that may be used to substitute for the example methods listed.

Changes in Ca²⁺ can be detected using fluorescent dyes (such as fura-2 and indo-1) (R. Y. Tsien, Nature 290, 527 (1981); R. Y. Tsien, T. Pozzan, T. J. Rink, J. Cell. Biol. 94, 325 (1982), the Ca²⁺ sensitive bioluminescent jellyfish protein aequorin (E. B. Ridgway and C. C. Ashley, Biochem. Biophys. Res. Commun. 29, 229 (1967), or Ca²⁺ sensitive microelectrodes (C. C. Ashley and A. K. Campbell, Eds., Detection and Measurement of Free Ca²⁺ in cells (Elsevier, North-Holland, Amsterdam, 1979).

An exemplary method for measuring intracellular calcium levels relies on calcium-sensitive fluorescent indicators. The choice of the appropriate calcium indicator, fluorescent, bioluminescent, metallochromic, or Ca²⁺-sensitive microelectrodes depends on the cell type and the magnitude and time constant of the event under study (Borle (1990) Environ Health Perspect 84:45-56). Calcium-sensitive indicators, such as fluo-3 and fura-2 (Molecular Probes, Inc., Eugene, Oreg.) are available as acetoxymethyl esters which are membrane permeable. When the acetoxymethyl ester form of the indicator enters a cell, the ester group is removed by cytosolic esterases, thereby trapping the free indicator in the cytosol. Interaction of the free indicator with calcium results in increased fluorescence of the indicator; therefore, an increase in the intracellular Ca²⁺ concentration of cells containing the indicator can be expressed directly as an increase in fluorescence (or an increase in the ratio of the fluorescence at two wavelengths when fura-2 is used). As an exemplary method of Ca²⁺ detection, cells could be loaded with the Ca²⁺ sensitive fluorescent dye fura-2 or indo-1, using standard methods, and any change in Ca²⁺ measured using a automated fluorescence detection system, which are known to one skilled in the art. Additionally, fluorescence imaging techniques can be utilized to visualize intracellular Ca²⁺ oscillations.

The intracellular free calcium concentration may be measured by microspectroflourometry using the fluorescent indicator dye Fura-2/AM (Bush et al, 1991). Stably transfected cells are seeded onto a 35 mm culture dish containing a glass coverslip insert. Cells are washed with HBS and loaded with 100 [L of Fura-2/AM (10 μM) for 20 to 40 min. After washing with HBS to remove the Fura-2/AM solution, cells are equilibrated in HBS for 10 to 20 min. Cells are then visualized under the 40× objective of a Leitz Fluovert FS microscope and fluorescence emission is determined at 510 nM with excitation wavelengths alternating between 340 nM and 380 nM. Raw fluorescence data are converted to calcium concentrations using standard calcium concentration curves and software analysis techniques.

In another method, the measurement of intracellular calcium can also be performed on a 96-well (or higher) format and with alternative calcium-sensitive indicators, preferred examples of these are: aequorin, Fluo-3, Fluo-4, Fluo-5, Calcium Green-1, Oregon Green, and 488 BAPTA. After activation of the receptors with agonist ligands the emission elicited by the change of intracellular calcium concentration can be measured by a luminometer, or a fluorescence imager; a preferred example of this is the fluorescence imager plate reader (FLIPR).

Cells expressing the receptor of interest are plated into clear, flat-bottom, black-wall 96-well plates (Costar) at a density of 80,000-150,000 cells per well and allowed to incubate for 48 hr at 5% CO₂, 37° C. The growth medium is aspirated and 100 μl of loading medium containing fluo-3 dye is added to each well. The loading medium contains: Hank's BSS (without phenol red)(Gibco), 20 mM HEPES (Sigma), 0.1 or 1% BSA (Sigma), dye/pluronic acid mixture (e.g. 1 mM Flou-3, AM (Molecular Probes) and 10% pluronic acid (Molecular Probes) mixed immediately before use), and 2.5 mM probenecid (Sigma)(prepared fresh). The cells are allowed to incubate for about 1 hour at 5% CO₂, 37° C.

During the dye loading incubation the compound plate is prepared. The compounds are diluted in wash buffer (Hank's BSS (without phenol red), 20 mM HEPES, 2.5 mM probenecid) to a 4×final concentration and aliquoted into a clear v-bottom plate (Nunc). Following the incubation the cells are washed to remove the excess dye. A Denley plate washer is used to gently wash the cells 4 times and leave a 100 μl final volume of wash buffer in each well. The cell plate is placed in the center tray and the compound plate is placed in the right tray of the FLIPR. The FLIPR software is setup for the experiment, the experiment is run and the data are collected. The data are then analyzed using an excel spreadsheet program.

FLIPR has shown considerable utility in measuring membrane potential of mammalian cells using voltage-sensitive fluorescent dyes but is useful for measuring essentially any cellular fluorescence phenomenon. The device uses low angle laser scanning illumination and a mask to selectively excite fluorescence within approximately 200 microns of the bottoms of the wells in standard 96 well plates.

The low angle of the laser reduces background by selectively directing the light to the cell monolayer. This avoids background fluorescence of the surrounding media. This system then uses a CCD camera to image the whole area of the plate bottom to measure the resulting fluorescence at the bottom of each well. The signal measured is averaged over the area of the well and thus measures the average response of a population of cells. The system has the advantage of measuring the fluorescence in each well simultaneously thus avoiding the imprecision of sequential measurement well by well measurement. The system is also designed to read the fluorescent signal from each well of a 96 or 384 well plate as fast as twice a second. This feature provides FLIPR with the capability of making very fast measurements in parallel. This property allows for the measurement of changes in many physiological properties of cells that can be used as surrogated markers to a set of functional assays for drug discovery. FLIPR is also designed to have state of the art sensitivity. This allows it to measure very small changes with great precision.

Antagonist ligands are identified by the inhibition of the signal elicited by agonist ligands.

“Dye” refers to a molecule or part of a compound that absorbs specific frequencies of light, including but not limited to ultraviolet light. The terms “dye” and “chromophore” are synonymous.

h. Promiscuous Second Messenger Assays

In recent years, “promiscuous” G proteins have increasingly been constructed with the aim of functionally coupling as many GPCRs as possible to the Ca²⁺ pathway and thus making them accessible for HTS screening. Promiscuity means the nonselectivity of the G protein for a GPCR. It is possible by means of molecular biological and biochemical methods to prepare promiscuous G proteins from hybrid G proteins or by mutagenesis within the mGluR family. Thus it is possible, for example, by fusion of the Gαi receptor recognition region to the Gαq effector activation region, to prepare a Gαq/i hybrid that receives signals from Gi-coupled receptors, but switches on the Gαq-PLC-β signal transduction pathway. A hybrid of this kind, in which the 5 C-terminal amino acids of Gαq had been replaced with the corresponding Gαi sequence (Gαqi5) was first described by Conklin et al., Nature 363, 274-276 (1993).

This “recoupling” of receptors has the advantage that the assay endpoint (increase in the intracellular Ca²⁺ concentration in comparison with adenylate cyclase inhibition) is more readily accessible through measurement methods and can be used in high throughput screening. The FLIPR (Molecular Devices) is an apparatus that typically measures intracellular Ca²⁺ levels in 96-well and 384-well formats. It is possible to coax receptors of different functional classes to signal through a pre-selected pathway through the use of promiscuous G. α. subunits. For example, by providing a cell based receptor assay system with an endogenously supplied promiscuous G.α subunit such as mGluR4 (please confirm) which might normally prefer to couple through a specific signaling pathway (e.g., G. i, Gq, Gₒ, etc.), can be made to couple through the pathway defined by the promiscuous G. α. subunit and upon agonist activation produce the second messenger associated with that subunit's pathway. In the case of mGluR4 this would involve activation of the G.q pathway and production of the second messenger phosphotidyl inositol. Through the use of similar strategies and tools, it is possible to bias receptor signaling through pathways producing other second messengers such as Ca⁺⁺, cAMP, and K⁺ currents, for example.

i. Transcriptional Reporters for Downstream Pathway Activation:

A reporter gene assay measures the activity of a gene's promoter. It takes advantage of molecular biology techniques, which allow one to put heterologous genes under the control of any promoter and introduce the construct into the genome of a mammalian cell (see, Gorman et al., Mol. Cell Biol. 2:1044-1051 (1982); Alam et al., Anal. Biochem. 188:245-254 (1990)). Activation of the promoter induces the expression of the reporter gene, as well as, or instead of, the endogenous gene. By design, the reporter gene codes for a reporter protein that can easily be detected and measured. Commonly, the reporter protein is a reporter enzyme activity that converts a commercially available substrate into a product. This conversion can be conveniently followed by direct optical measurement and may allow for the quantification of the amount of reporter enzyme activity produced.

Reporter genes are commercially available on a variety of plasmids for the study of gene regulation in a large variety of organisms (see, Alam et al., supra, 1990). Promoters of interest can be inserted into multiple cloning sites provided for this purpose in front of the reporter gene on a plasmid (see, Rosenthal, Methods Enzymol. 152:704-720 (1987); Shiau et al., Gene 67:295-299 (1988)). Standard techniques are used to introduce these reporter genes into a cell type or whole organism (such as described in Sambrook et al. Molecular cloning, Cold Spring Harbor Laboratory Press (1989)). Resistance markers provided on a plasmid can then be used to select for successfully transfected cells.

“Reporter gene” means a gene that encodes a reporter enzyme, such as they are known in the art or are later developed, such as a reporter enzyme activity. “Reporter enzyme” means an enzyme that encode a reporter enzyme that has a detectable read-out, such as β-lactamase, β-galactosidase, or luciferase (for β-lactamase, see WO 96/30540 to Tsien, published Oct. 3, 1996). Reporter enzymes can be detected using methods known in the art, such as the use of chromogenic or fluorogenic substrates for reporter enzymes as such substrates are known in the art. Such substrates are preferably membrane permeant. Chromogenic or fluorogenic readouts can be detected using, for example, optical methods such as absorbance or fluorescence. A reporter gene can be part of a reporter gene construct, such as a plasmid or viral vector, such as a retrovirus or adeno-associated virus. A reporter gene can also be extra-chromosomal or be integrated into the genome of a host cell. The expression of the reporter gene can be under the control of exogenous expression control sequences or expression control sequences within the genome of the host cell. Under the latter configuration, the reporter gene is preferably integrated into the genome of the host cell.

“Reporter enzyme activity” refers to the activity of a reporter enzyme in a membrane compartment and includes background reporter enzyme activity and de novo reporter enzyme activity. “Background reporter enzyme activity” refers to a reporter enzyme activity that exists in a membrane compartment that was not made in response to a stimulus, such as a test chemical. A background reporter enzyme activity and a de novo reporter enzyme activity can be the same enzyme activity, such as β-lactamase activity. In such instances, background reporter enzyme activity can be referred to as “noise” and de novo reporter enzyme.

“Reporter β-lactamase” refers to a β-lactamase that is inhibited by a β-lactamase inhibitor, whereas an “inhibitor resistant β-lactamase” refers to a P-lactamase whose activity is inhibited less by a given β-lactamase inhibitor than a reporter β-lactamase. In such instances, the activity of the reporter β-lactamase will be inhibited at a greater rate by a β-lactamase inhibitor than will the activity of an inhibitor resistant β-lactamase. Preferably, the inhibitor resistant β-lactamase can degrade a β-lactamase inhibitor in such a way that the reporter β-lactamase activity is not inhibited by the β-lactamase inhibitor. Preferably, such β-lactamase inhibitors bind to the catalytic site of both the reporter β-lactamase and the inhibitor resistant β-lactamase. Most preferably, the β-lactamase activity is an irreversible inhibitor of the reporter β-lactamase. Preferred reporter β-lactamases have sequences such as set forth in WO 96/30540 to Tsien et al., issued Apr. 21, 1998.

The intracellular signal initiated by binding of an agonist to a cell surface receptor, e.g., mGluR, sets in motion a cascade of intracellular events, the ultimate consequence of which is a rapid and detectable change in the transcription or translation of one or more genes. The activity of the receptor can therefore be monitored by measuring the expression of a reporter gene driven by control sequences responsive to human metabotropic glutamate receptor activation.

In a preferred reporter gene assay, the reporter gene, associated with or without a promoter, is transfected into cells, either transiently or stably. Activation of the reporter gene by, for examiner, the activation of a receptor, leads to a change in reporter enzyme activity levels via transcriptional and translational events. The amount of reporter activity enzyme present can be measured via its enzymatic action on a substrate. The substrate can be a small uncharged molecule that, when added to the extracellular solution, can penetrate the plasma membrane to encounter the reporter enzyme activity. A charged molecule can also be employed, but the charges can be masked by groups that will be cleaved by endogenous cellular enzymes (e.g., esters cleaved by cytoplasmic esterases).

To achieve the high sensitivity in a reporter enzyme activity assay one has to maximize the amount of signal generated by a single reporter enzyme. An optimal reporter enzyme activity will convert 10⁵ substrate molecules per second under saturating conditions (see, Stryer, Introduction to enzymes. In Biochemistry, New York, W. H. Freeman and Co. (1981), pp. 103 to 134). β-lactamases will cleave about 10³ molecules of their preferred substrates per second (Chang et al., Proc. Natl. Acad. Sci. USA 87:2823-2827 (1990)). Using a fluorogenic substrate one can obtain up to 10⁶ photons per fluorescent product produced, depending on the type of dye used, when exciting with light of the appropriate wavelength. The signal terminates with the bleaching of the fluorophore (Tsien et al., Handbook of Biological Confocal Microscopy, ed: Pawley, J. B. Plenum Publishing Co (1990), pp. 169-178). These numbers illustrate the theoretical magnitude of signal obtainable in this type of measurement. In practice, a minute fraction of the photons generated will be detected, but this holds true for fluorescence, bioluminescence or chemiluminescence. A good fluorogenic substrate for a reporter enzyme activity should have a high turnover at the enzyme in addition to good optical properties such as high extinction and high fluorescence.

As used herein “promoter” refers to the transcriptional control elements necessary for receptor-mediated regulation of gene expression, including not only the basal promoter, but also any enhancers or transcription-factor binding sites necessary for receptor-regulated expression. By selecting promoters that are responsive to the intracellular signals resulting from agonist binding, and operatively linking the selected promoters to reporter genes whose transcription, translation or ultimate activity is readily detectable and measurable, the transcription based reporter assay provides a rapid indication of whether a given receptor is activated. Preferred reporter genes are those that are readily detectable. The reporter gene may also be included in the construct in the form of a fusion gene with a gene that includes desired transcriptional regulatory sequences or exhibits other desirable properties. Examples of reporter genes include, but are not limited to CAT (chloramphenicol acetyl transferase) (Alton and Vapnek (1979), Nature 282: 864-869) luciferase, and other enzyme detection systems, such as β-galactosidase; firefly luciferase (deWet et al. (1987), Mol. Cell. Biol. 7:725-737); bacterial luciferase (Engebrecht and Silverman (1984), PNAS 1: 4154-4158; Baldwin et al. (1984), Biochemistry 23: 3663-3667); alkaline phosphatase (Toh et al. (1989) Eur. J. Biochem. 182: 231-238, Hall et al. (1983) J. Mol. Appl. Gen. 2: 101), human placental secreted alkaline phosphatase (Cullen and Malim (1992) Methods in Enzymol. 216:362-368). All of these genes are wll known to one skilled in the art as are assays for the detection of their products.

Genes particularly well suited for monitoring receptor activity are the “immediate early” genes, which are rapidly induced, generally within minutes of contact between the receptor and the effector protein or ligand. The induction of immediate early gene transcription does not require the synthesis of new regulatory proteins. In addition to rapid responsiveness to ligand binding, characteristics of preferred genes useful to make reporter constructs include: low or undetectable expression in quiescent cells; induction that is transient and independent of new protein synthesis; subsequent shut-off of transcription requires new protein synthesis; and mRNAs transcribed from these genes have a short half-life. It is preferred, but not necessary that a transcriptional control element have all of these properties for it to be useful.

Transcription-based reporter assays can be used to test functional ligand-receptor or ligand-ion channel interactions for categories of cell surface-localized receptors including, but not limited to ligand-gated ion channels and voltage-gated ion channels, G protein-coupled receptors and growth factor receptors. Examples of each group include, nut are not limited to:

a) ligand-gated ion channels: nicotinic acetylcholine receptors, GABA (γ-aminobutyric acid) receptors, excitatory receptors (e.g., glutamate and aspartate), and the like;

b) voltage-gated ion channels: calcium channels, potassium channels, sodium channels, NMDA receptor (actually a ligand-gated, voltage-dependent ion channel) and the like;

c) G protein-coupled receptors: adrenergic receptors, muscarinic receptors and the like and

d) Growth factor receptors (Both RTKs and non-RTKs): Nerve growth factor NGF, heparin binding growth factors and other growth factors.

Transcriptional control elements include, but are not limited to, promoters, enhancers, and repressor and activator binding sites. Suitable transcriptional regulatory elements may be derived from the transcriptional regulatory regions of genes whose expression is rapidly induced, generally within minutes of contact between the cell surface protein and the effector protein that modulates the activity of the cell surface protein. Immediate early genes are genes that are rapidly induced upon binding of a ligand to a cell surface protein. The induction of immediate early gene transcription does not require the synthesis of new regulatory proteins. The transcriptional control elements that are preferred for use in the gene constructs include transcriptional control elements from immediate early genes, elements derived from other genes that exhibit some or all of the characteristics of the immediate early genes, or synthetic elements that are constructed such that genes in operative linkage therewith exhibit such characteristics. The characteristics of preferred genes from which the transcriptional control elements are derived include, but are not limited to, low or undetectable expression in quiescent cells, rapid induction at the transcriptional level within minutes of extracellular simulation, induction that is transient and independent of new protein synthesis, subsequent shut-off of transcription requires new protein synthesis, and mRNAs transcribed from these genes have a short half-life. It is not necessary for all of these properties to be present.

Examples of such genes include, but are not limited to, the immediate early genes (see, Sheng et al. (1990) Neuron 4: 477-485), such as c-fos, which is responsive to a number of different stimuli is the c-fos proto-oncogene. The c-fos gene is activated in a protein-synthesis-independent manner by growth factors, hormones, differentiation-specific agents, stress, and other known inducers of cell surface proteins. The induction of c-fos expression is extremely rapid, often occurring within minutes of receptor stimulation. This characteristic makes the c-fos regulatory regions particularly attractive for use as a reporter of receptor activation.

The c-fos regulatory elements include (see, Verma et al., 1987, Cell 51: 513-514): a TATA box that is required for transcription initiation; two upstream elements for basal transcription, and an enhancer, which includes an element with dyad symmetry and which is required for induction by TPA, serum, EGF, and PMA. The 20 bp c-fos transcriptional enhancer element located between −317 and −298 bp upstream from the c-fos mRNA cap site, is essential for serum induction in serum starved NIH 3T3 cells. One of the two upstream elements is located at −63 to −57 and it resembles the consensus sequence for cAMP regulation.

More, the transcription factor CREB (cyclic AMP responsive element binding protein) is, as the name implies, responsive to levels of intracellular cAMP. Therefore, the activation of a receptor that signals via modulation of cAMP levels can be monitored by measuring either the binding of the transcription factor, or the expression of a reporter gene linked to a CREB-binding element (termed the CRE, or cAMP response element). The DNA sequence of the CRE is known. Reporter constructs responsive to CREB binding activity are described in U.S. Pat. No. 5,919,649.

Still other promoters and transcriptional control elements, in addition to the c-fos elements and CREB-responsive constructs, include the vasoactive intestinal peptide (VIP) gene promoter (cAMP responsive; Fink et al., 1988, Proc. Natl. Acad. Sci. 85:6662-6666); the somatostatin gene promoter (cAMP responsive; Montminy et al., 1986, Proc. Natl. Acad. Sci. 8.3:6682-6686); the proenkephalin promoter (responsive to cAMP, nicotinic agonists, and phorbol esters; Comb et al., 1986, Nature 323:353-356); the phosphoenolpyruvate carboxy-kinase (PEPCK) gene promoter (cAMP responsive; Short et al., 1986, J. Biol. Chem. 261:9721-9726).

Additional examples of transcriptional control elements that are responsive to changes in GPCR activity include, but are not limited to those responsive to the AP-1 transcription factor and those responsive to NF-.kappa.B activity. The consensus AP-1 binding site is the palindrome TGA(C/G)TCA (Lee et al., 1987, Nature 325: 368-372; Lee et al., 1987, Cell 49: 741-752). The AP-1 site is also responsible for mediating induction by tumor promoters such as the phorbol ester 12-O-tetradecanoylphorbol-β-acetate (TPA), and are therefore sometimes also referred to as a TRE, for TPA-response element. AP-1 activates numerous genes that are involved in the early response of cells to growth stimuli.

The consensus sequence NF-.kappa.B binding element is well known. A large number of genes have been identified as NF-.kappa.B responsive, and their control elements can be linked to a reporter gene to monitor GPCR activity. Examples of genes responsive to NF-.kappa.B are known to one skilled in the art. See, Hiscott et al., 1993, Mol. Cell. Biol. 13: 6231-6240 which discusses IL-β; Shakhov et al., 1990, J. Exp. Med. 171: 35-47, that discusses TNF α, etc. Each of these references is incorporated herein by reference. Vectors encoding NF-.kappa.B-responsive reporters are also known in the art or can be readily made by one of skill in the art using, for example, synthetic NF-KB elements and a minimal promoter, or using the NF-.kappa.B-responsive sequences of a gene known to be subject to NF-KB regulation. Further, NF-.kappa.B responsive reporter constructs are commercially available from, for example, CLONTECH.

A given promoter construct should be tested by exposing mGluR and Glast1-expressing cells, transfected with the construct, to a ligand, e.g., glutamate or a modulating moiety under investigation. An increase of at least two-fold in the expression of reporter in response to the known or unknown ligand indicates that the reporter is an indicator of receptor, e.g., mGluR activity.

In a representative embodiment, the step of detecting interaction of a ligand and its corresponding cell surface receptor, e.g., mGluR protein comprises detecting, in a cell-based assay, change(s) in the level of expression of a gene controlled by a transcriptional regulatory sequence responsive to signaling by the mGluR polypeptide. Reporter gene based assays of this invention measure the end stage of the above described cascade of events, e.g., transcriptional modulation. Accordingly, in practicing one embodiment of the assay, a reporter gene construct is inserted into the reagent cell in order to generate a detection signal dependent on mGluR mediated signaling. Expression of the reporter gene, thus, provides a valuable screening tool for the development of compounds that act as agonists or antagonists of mGluR-dependent signal induction.

In practicing one embodiment of the assay, a reporter gene construct is inserted into the reagent cell in order to generate a detection signal dependent on second messengers generated by the target cell receptor-dependent induction with a modulating moiety. Typically, the reporter gene construct will include a reporter gene in operative linkage with one or more transcriptional regulatory elements responsive to activation of a metabotropic glutamate receptor, with the level of expression of the reporter gene providing the cell surface receptor-dependent detection signal. The amount of transcription from the reporter gene may be measured using any method known to those of skill in the art to be suitable. For example, mRNA expression from the reporter gene may be detected using RNAse protection or RNA-based PCR, or the protein product of the reporter gene may be identified by a characteristic stain or an intrinsic activity. The amount of expression from the reporter gene is then compared to the amount of expression in either the same cell in the absence of the test compound or it may be compared with the amount of transcription in a substantially identical cell that lacks the target receptor protein. Any statistically or otherwise significant difference in the amount of transcription indicates that the test compound has in some manner altered the inductive activity of the target cell receptor protein.

As described in further detail below, in preferred embodiments the gene product of the reporter is detected by an intrinsic activity associated with that product. For instance, the reporter gene may encode a gene product that, by enzymatic activity, gives rise to a detection signal based on color, fluorescence, or luminescence. Many reporter genes are known to those of skill in the art and others may be identified or synthesized by methods known to those of skill in the art. A reporter gene includes any gene that expresses a detectable gene product, which may be RNA or protein.

Consequently, in a broad aspect, the subject drug screening assays of the present invention provides a recombinant cell, e.g., for carrying out certain of the drug screening methods above, comprising: (i) an expressible recombinant gene encoding a heterologous cell surface polypeptide whose signal transduction activity is modulated by binding to an agonist, e.g., glutamate; and (ii) a reporter gene construct containing a reporter gene in operative linkage with one or more transcriptional regulatory elements responsive to the signal transduction activity of the cell surface receptor protein.

In furtherance of the above, in order to assay mGluR activity with a glutamate-responsive transcriptional reporter construct, cells that stably express the mGluR protein are stably transfected with the reporter construct, with the proviso that cells also express mGlAST. To screen for agonists, the cells are left untreated, exposed to candidate modulators, or exposed to glutamate, and expression of the reporter is measured. The glutamate-treated cultures serve as a standard for the level of transcription induced by a known agonist. An increase of at least 50% in reporter expression in the presence of a candidate modulator indicates that the candidate is a modulator of mGluR activity. An agonist will induce at least as much, and preferably the same amount or more, reporter expression than the glutamate. This approach can also be used to screen for inverse agonists where cells express a mGluR protein at levels such that there is an elevated basal activity of the reporter in the absence of glutamate or another agonist. A decrease in reporter activity of 10% or more in the presence of a candidate modulator, relative to its absence, indicates that the compound is an inverse agonist.

To screen for antagonists, the cells co-expressing one or more mGluR subtypes and a GLAST protein and carrying the reporter construct are exposed to glutamate (or another agonist such as a glutamate analogue) in the presence and absence of candidate modulator. A decrease of 10% or more in reporter expression in the presence of candidate modulator, relative to the absence of the candidate modulator, indicates that the candidate is a modulator of mGluR activity.

Controls for transcription assays include cells not expressing the target receptor, e.g., mGluR but carrying the reporter construct, as well as cells with a promoterless reporter construct. Compounds that are identified as modulators of mGluR-regulated transcription should also be analyzed to determine whether they affect transcription driven by other regulatory sequences and by other receptors, in order to determine the specificity and spectrum of their activity.

The transcriptional reporter assay, and most cell-based assays, are well suited for screening expression libraries for proteins for those that modulate mGluR activity. The libraries can be, for example, cDNA libraries from natural sources, e.g., plants, animals, bacteria, etc., or they can be libraries expressing randomly or systematically mutated variants of one or more polypeptides. Genomic libraries in viral vectors can also be used to express the mRNA content of one cell or tissue, in the different libraries used for screening of mGluR expressing cell.

Any of the assays of receptor activity, including the GTP-binding, GTPase, adenylate cyclase, cAMP, phospholipid-breakdown, diacylglyceorl, inositol triphosphate, PKC, kinase and transcriptional reporter assays, can be used to determine the presence of an agent in a sample, e.g., a tissue sample, that affects the activity of the mGluR receptor molecule. To do so, cell preparations of the invention, i.e., those co-expressing one or more metabotropic glutamate receptor subtypes and a transport protein exemplified by GLAST are assayed for activity in the presence and absence of the sample or an extract of the sample or compared to cell not expressing the mGluR subtype. An increase in mGluR activity in the presence of the sample or extract relative to the control cells indicates that the sample contains an agonist of the receptor activity. A decrease in receptor activity in the presence of, for example, glutamate or another agonist and the sample, relative to receptor activity in the presence of glutamate alone indicates that the sample contains an antagonist of mGluR activity. If desired, samples can then be fractionated and further tested to isolate or purify the agonist or antagonist. The amount of increase or decrease in measured activity necessary for a sample to be said to contain a modulator depends upon the type of assay used. Generally, a 10% or greater change (increase or decrease) relative to an assay performed in the absence of a control cell preparation or sample indicates the presence of a modulator in the sample. One exception is the transcriptional reporter assay, in which at least a two-fold increase or 10% decrease in signal is necessary for a sample to be said to contain a modulator. It is preferred that an agonist stimulates at least 50%, and preferably 75% or 100% or more, e.g., 2-fold, 5-fold, 10-fold or greater receptor activation than with glutamate alone or cells not expressing the cell surface receptor.

Other functional assays include, for example, microphysiometer or biosensor assays (see Hafner, 2000, Biosens. Bioelectron. 15: 149-158, incorporated herein by reference).

Modulation of Human Metabotropic Glutamate Receptor Activity in a Cell According to the Invention

The discovery of glutamate as a ligand of various human CNS related receptors provides methods of identifying modulators of one or more of the several types of calcium-permeable CNS ion channels such as : a) the voltage-dependent Ca²⁺ channels; and b) other channels directly coupled to glutamate (or excitatory amino acid) receptors. Such channels are reviewed in: Sommer, B. and Seeburg, P. H. “Glutamate receptor channels: novel properties and new clones” Trends Pharmacological Sciences 13:291-296 (1992); Nakanishi, S., “Molecular Diversity of glutamate receptors and implications for brain function”, Science 248:597-603 (1992).

As an endogenous neurotransmitter, L-glutamate interacts with several different proteins during the course of synaptic transmission. These interactions include the multiple receptors mediating synaptic responses as well as the transport system that is responsible for clearing L-glutamate from the synaptic cleft and terminating its excitatory signal. In developing the assays of the invention, it was recognized that endogenous glutamate produced and secreted from cultured cells interferes with the ability to measure a functional response of metabotropic glutamate receptors coupled to a reporter based system. In fact, high basal levels of reporter gene expression are observed in the absence of a glutamate transport protein arising form activation of recombinantly expressed mGluR receptors by the endogenous glutamate. The invention relies on the discovery that co-expression of a cell surface protein, e.g., mGluR with a glutamate transporter protein is effective to remove the extracellular glutamate from the media allowing the ability to measure mGluR activation in direct response to a modulating moiety.

Modulating moieties for use in the preferred assays of the invention include agents include glutamates as defined herein, as well as additional modulators identified using the screening methods described herein. In general, modulating metabotropic glutamate receptor activity causes an increase or decrease in a cellular response which occurs upon metabotropic glutamate receptor activation. Cellular responses to metabotropic glutamate receptor activation vary depending upon the type of metabotropic glutamate receptor activated.

Consequently, modulation of metabotropic glutamate receptor activity can be used to produce different effects such as anticonvulsant effects, neuroprotectant effects, analgesic effects, cognition-enhancement effects, and muscle-relaxation effects, each of which has therapeutic applications. Thus, one important application of this aspect of the present invention is in drug screening where rapid methods of testing for the activity of test compounds are needed. The use of cells of the present invention which co-express both at least one human metabotropic glutamate receptor subtype involved in the modulation of intracellular calcium concentration and a transporter protein such as GLAST provides methods whereby compounds can be tested for their effect on the release of intracellular calcium. The sensitivity of the system as well as the high signal to noise allows cells in small volumes to be screened. Furthermore, the availability of luminometers that measure cells in microtiter plates provides for testing of thousands of compounds for agonist or antagonist activity. For example, a mammalian cell line transfected with a gene coding for a metabotropic glutamate receptor subtype which activates intracellular calcium release in cells and also expressing a glutamate transport protein may be used to study the effect of drugs on the release of intracellular calcium stimulated by the metabotropic glutamate receptor. Compounds used therapeutically should have minimal side effects at therapeutically effective doses.

Identifying Receptor Agonists

The invention thus provides, in several aspects assays that can be used to identify receptor agonists. A receptor agonist is any molecule that specifically interacts with a receptor and initiates a biological response mediated by that receptor. For example, an agonist for receptor X can be any molecule that induces an X receptor-mediated response in an X receptor-specific manner. Thus, a metabotropic glutamate receptor agonist is any molecule that specifically interacts with a metabotropic glutamate receptor and initiates a mGluR receptor-mediated response.

Such assays involve monitoring at least one of the biological responses mediated by a mGluR receptor. Consequently, activation of a particular metabotropic glutamate receptor refers to the production of one or more activities associated with the type of receptor activated, for example: (1) activation of phospholipase C, (2) increases in phosphoinositide (PI) hydrolysis, (3) intracellular calcium release, (4) activation of phospholipase D, (5) activation or inhibition of adenylyl cyclase, (6) increases or decreases in the formation of cyclic adenosine monophosphate (cAMP), (7) activation of guanylyl cyclase, (8) increases in the formation of cyclic guanosine monophosphate (cGMP), (9) activation of phospholipase A₂, (10) increases in arachidonic acid release, and (11) increases or decreases in the activity of ion channels, for example voltage- and ligand-gated ion channels. Inhibition of metabotropic glutamate receptor activation (antagonist) on the other hand prevents one or more of these activities from occurring.

The specificity of the interactions of receptor agonists with metabotropic glutamate receptors as well as other receptors coupled to glutamate can be determined, for example by the use of a known antagonist. For example, a test molecule that induces a biological response that a metabotropic glutamate receptor mediates can be identified as a metabotropic glutamate receptor agonist if a metabotropic glutamate receptor antagonist inhibits the induction of that particular biological response. In addition, the specificity of agonist-receptor interactions can be demonstrated using heterologous expression systems, receptor binding analyses, or any other method that provides a measure of agonist-receptor interaction.

Thus, in an exemplary embodiment, a metabotropic glutamate receptor agonist can be identified by contacting positive cells, vis-à-vis cells co-expressing one or more recombinant metabotropic glutamate receptor subtype and a glutamate transport protein, e.g.,GLAST with a test molecule, and determining if that test molecule induces a mGluR response in those cells in a mGluR specific manner. A test molecule can be any molecule having any chemical structure and comparing the response to a test cell population, wherein the cells do not express a metabotropic glutamate receptor, wherein an increase in second messenger activity in the positive cells relative to the test cells (negative cells wherein the cells do not express a functional target receptor protein) suggest that the unknown test agent is an agonist of the target cell receptor.

Intracellular calcium concentrations can be monitored using any method. A preferred aspect of the invention provides for the establishment of a calcium mobilization assay using cell co-expressing one or more metabotropic glutamate receptor proteins in conjunction with a glutamate transport protein to identify novel molecules that antagonize calcium mobilization in these cells. These assays may take several forms but are generally modeled after use of a calcium responsive fluorescent dye (such as Fura-2) that detects calcium ions. In this case, cells are loaded with fura-2, a fluorescent dye, and monitored by dual emission microfluorimetry. The fura-2 loading process can involve washing the cells (e.g., one to four times) with incubation medium lacking calcium. This medium can be balanced with sucrose to maintain osmolarity. After washing, the cells can be incubated (e.g., 30 minutes) with loading solution. This loading solution can contain, for example, 5 μM fura-2/AM and a non-ionic/non-denaturing detergent such as Pluronic F-127. The non-ionic/non-denaturing detergent can help disperse the acetoxymethyl (AM) esters of fura-2. After incubation with the loading solution, the cells can be washed (e.g., one to four times) with, for example, PBS without calcium or magnesium to remove extracellular dye.

Once loaded, the intracellular calcium concentration ([Ca²⁺]i) can be calculated from the fluorescence ratio (340 and 380 nm excitation and 510 nm emission wavelength) according to the following equation:
[Ca²⁺]i=(R−Rₘᵢₙ)k_{d}β/(Rₘₐₓ−R); where R=fluorescence ratio recorded from cell;
Rₘᵢₙ=fluorescence ratio of fura-2 free acid recorded in absence of Ca²⁺; Rₘₐₓ=fluorescence ratio of fura-2 free acid recorded in saturating concentration of Ca²⁺; k_{d}=calcium dissociation constant of the dye; and, β- the ratio of fluorescence of fura-2 free acid in the Ca²⁺ free form to the Ca²⁺ saturated form recorded at the wavelength used in the denominator of the ratio. Using an image processing system such as a COMPIX C-640 SIMCA (Compix Inc., Mars, Pa.) system with an inverted microscope, images can be acquired for analysis every 0.4 seconds.
Identifying Receptor Antagonists

A receptor antagonist is any molecule that specifically interacts with a receptor and inhibits a receptor agonist from initiating a biological response mediated by that receptor. For example, an antagonist for receptor X can be any molecule that inhibits an X receptor agonist from inducing an X receptor-mediated response in an X receptor-specific manner. Thus, a mGluR receptor antagonist is any molecule that specifically interacts with a mGluR receptor and inhibits a mGluR agonist from initiating a mGluR receptor-mediated response.

For example, a mGluR receptor antagonist can be identified by contacting mGluR receptor positive cells ( cell co-expressing one or more metabotropic glutamate receptor subtypes and a glutamate transport protein (GLAST)) with a mGluR receptor agonist such as glutamate or an analogue thereof and a test molecule, and determining if that test molecule inhibits the mGluR receptor agonist from inducing a mGluR receptor response in those cells in a mGluR receptor-specific manner. Again, a test molecule can be any molecule having any chemical structure. For example, a test molecule can be a polypeptide, or a chemical entity.

It is to be understood that each of the assays for identifying receptor agonists described herein can be easily adapted such that receptor antagonists can be identified.

The agent (modulating moiety or test compound) can be delivered to a cell by adding it to culture medium. The amount to deliver will vary with the identity of the agent and with the purpose for which it is delivered. For example, in a culture assay to identify antagonists of human metabotropic glutamate receptor activity, one will preferably add an amount of glutamate that half-maximally activates the receptors (e.g., approximately EC₅₀), preferably without exceeding the dose required for receptor saturation. This dose can be determined by titrating the amount of glutamate to determine the point at which further addition of glutamate has no additional effect on human metabotropic glutamate receptor activity.

Identifying Allosteric Modulators:

A “potentiators” can be any material which improves or increases the efficacy of the pharmaceutical composition and generally binds to the target cell surface receptor, e.g., metabotropic glutamate receptor at a site other than the ligand binding site.

For allosteric screening, cells co-expressing a human metabotropic glutamate receptor protein (hmGluR) and a GLAST protein or membranes isolated from them are used in a functional assay that measures an activity of the receptor in the presence and absence of a candidate compound. Inverse agonists are those compounds that reduce the constitutive activity of the receptor by at least 10%.

Candidate Modulators Useful According to the Invention

In another aspect, the invention encompasses a modulator of a cell surface receptor protein, e.g., human mGluR. The candidate compound a/k/a modulating moiety may be a synthetic compound, or a mixture of compounds, or may be a natural product (e.g. a plant extract or culture supernatant). A candidate compound according to the invention includes a small molecule that can be synthesized, a natural extract, peptides, proteins, carbohydrates, lipids etc.

Candidate modulators can be screened from large libraries of synthetic or natural compounds. Numerous means are currently used for random and directed synthesis of saccharide, peptide, lipid, carbohydrate, and nucleic acid based compounds. Synthetic compound libraries are commercially available from a number of companies including, for example, Maybridge Chemical Co. (Trevillet, Cornwall, UK), Comgenex (Princeton, N.J.), Brandon Associates (Merrimack, N.H.), and Microsource (New Milford, Conn.). A rare chemical library is available from Aldrich (Milwaukee, Wis.). Combinatorial libraries of small organic molecules are available and can be prepared. Alternatively, libraries of natural compounds in the form of bacterial, fungal, plant and animal extracts are available from e.g., Pan Laboratories (Bothell, Wash.) or MycoSearch (NC), or are readily produceable by methods well known in the art. Additionally, natural and synthetically produced libraries and compounds are readily modified through conventional chemical, physical, and biochemical means.

As noted previously herein, candidate modulators can also be variants of known polypeptides (e.g., glutamate, antibodies) or nucleic acids (e.g., aptamers) encoded in a nucleic acid library. Cells (e.g., bacteria, yeast or higher eukaryotic cells) transformed with the library can be grown and prepared as extracts, which are then applied in human metabotropic glutamate receptor binding assays or functional assays of human metabotropic glutamate receptor activity.

Prior to therapeutic use in a human, the compounds are preferably tested in vivo using animal models. Animal studies to evaluate a compound's effectiveness to treat different diseases or disorders, or exert an effect such as an analgesic effect, a cognition-enhancement effect, or a muscle-relaxation effect, can be carried out using standard techniques.

When a modulator of human metabotropic glutamate receptor activity is administered to an animal for the treatment of a disease or disorder, the amount administered can be adjusted by one of skill in the art on the basis of the desired outcome. Successful treatment is achieved when one or more measurable aspects of the pathology (e.g., tumor cell growth, accumulation of inflammatory cells) is changed by at least 10% relative to the value for that aspect prior to treatment.

High-Throughput-Screening-Calcium Assay:

High-throughput screening allows a large number of molecules to be tested. For example, a large number of molecules can be tested individually using rapid automated techniques or in combination with using a combinatorial library of molecules. Individual compounds able to modulate a target receptor activity present in a combinatorial library can be obtained by purifying and retesting fractions of the combinatorial library. Thus, thousands to millions of molecules can be screened in a short period of time. Active molecules can be used as models to design additional molecules having equivalent or increased activity.

In the case of metabotropic glutamate receptor modulators, high-throughput screening of chemical libraries using cells stably transfected with individual, cloned mGluRs may offer a promising approach to identify new lead compounds which are active on the individual receptor subtypes. Knopfel et al. (1995), J. Med. Chem. 38:1417. These lead compounds could serve as templates for extensive chemical modification studies to further improve potency, mGluR subtype selectivity, and important therapeutic characteristics such as bioavailability. Active molecules can be used as models to design additional molecules having equivalent or increased activity. Preferably, the activity of molecules in different cells may be tested to identify a metabotropic glutamate receptor agonist or metabotropic glutamate receptor antagonist molecule which mimics or blocks one or more activities of glutamate at a first type of metabotropic glutamate receptor

One approach for developing a high through-put functional GPCR assay is the use of reporter gene constructs. Reporter gene constructs couple transcriptional enhancers that are regulated by various intracellular second messengers with appropriate promoter and reporter gene elements to produce a surrogate signal transduction system responsive to signaling pathways activated by various hormone receptors (Deschamps, Science, 1985 230 :1174-7; Montminy, Proc. Nail Acad Sci USA, 1986 83 :6682-6686; Angel, Cell, 1987, 49:729-39 ; Fisch, Mol. Cell Biol, 1989 9:1327-31).However, data generated by conventional high-throughput systems for measuring, for example, glutamate mediated signal transduction are contaminated by endogenous glutamate, which is produced and secreted from cultured cells. It is believed that this endogenous glutamate interferes with the ability to measure a true functional response of metabotropic glutamate receptors coupled to a reporter gene system. Specifically, the endogenous production of glutamate has been linked to high basal levels of reporter gene expression arising form activation of recombinantly expressed mGluR receptors by the endogenous glutamate.

While the mainstream of the pharmaceutical industry is moving to solve HTS throughput problems, e.g., by developing multi-well plates with more, and thus smaller, individual wells per plate, current models are still plagues by high-basal levels of reporter gene expression. This drawback is in addition to the expenditure of untold millions of dollars to achieve probably less than an order of magnitude increase in speed without other significant technological advantages which would increase the information content of the screening process.

Therefore there is a need for methods to assay the effects of compounds on the function of biological targets, exemplified by G-protein coupled receptors. In particular, there exists a need to identify modulators of metabotropic glutamate receptors for use in developing novel strategies for a variety of psychiatric and neurological disorders. It would be a further advancement to provide methods for screening for agonists, antagonists, and modulatory molecules that act on such receptors.

The assays of the present invention particularly include high-throughput screening assays. Apparatuses for quantitating simultaneously measurements from a multitude of samples are known in the art. For example, as noted, supra, the Fluorometric Imaging Plate Reader (FLIPR), available from Molecular Devices, is useful for single wavelength detection of changes in intracellular calcium or sodium, membrane potential and pH. The FLIPR works best with the visible wavelength calcium indicators, Fluo-3 and Calcium green-1. Both of these dyes have been used successfully for the HTS assay, but Fluo-3 being preferred. The apparatus and reader can be programmed to simultaneously deliver compounds to and image all 96 wells of a microplate within one second, and is therefore amendable to high throughput formats. This technology allows the measurement of the intracellular calcium mobilization in cells attached to the bottom of a 96 well plate An argon-ion laser excites a fluorescent indicator dye suitable for the specific change being measured, and the emitted light is detected using the associated optical system. A camera system then images the entire plate and integrates data over a time interval specified by the user.

For antagonist studies, FLIPR obtains a baseline fluorescence for about.30 sec, then it adds the compounds to all 96 wells simultaneously and begins to monitor changes in intracellular Ca²⁺. After 2 min, the contents or the agonist plate is added to the cells. The maximal Ca²⁺ response (in optical units) for 1 nM C3a (???) in the presence of vehicle (100%) or the various concentrations of compound is determined. Inhibition curves were generated essentially as described for the single cuvette Fura-2 assay. Typically 4 uM Fluo-3 is loaded into the cells for 1 hr at 37° C. in cell media without fetal calf serum and with 1.5 mM sulfinpyrazone to inhibit dye release from the cells. The media is aspirated from the cells and fresh media is added for 10 min at 37° C. to allow hydrolysis of the dye and remove extracellular dye. The media is thereafter aspirated and replaced with KRH buffer. After 10 min at 37° C. the cells are placed in FLIPR apparatus for analysis.

Alternatively, apparatuses such as the Voltage ion Probe Reader (VIPR) available from Aurora Biosciences may be used for dual wavelength detection of fluorescence resonance energy transfer (FRET) between two fluorescent molecules. FRET is a distance-dependent interaction between the electronic excited states of two dye molecules, and may be used to investigate a variety of biological events that produce changes in molecular proximity, including the activity of Na⁺, K⁺, Cl⁻, Ca²⁺, and Ligand-gated Ion Channels. The VIPR reader is amenable to both 96- and 3iB4-well formats.

High Throughput Screening Kit

A high throughput screening kit according to the invention comprises all the necessary means and media for performing the detection of a modulator compound including an agonist, antagonist, inverse agonist or inhibitor to the receptor of the invention in the presence of glutamate, preferably at a concentration in the range of 1 nM to 10 μM. The kit comprises the following successive steps. Recombinant cells of the invention, comprising and co-expressing the nucleotide one or more human metabotropic glutamate receptor proteins and a transport protein, e.g., GLAST, are grown on a solid support, such as a microtiter plate, more preferably a 96 well microtiter plate, according to methods well known to the person skilled in the art especially as described in WO 00/02045. Modulating moieties or compounds according to the invention, at concentrations from about 1 nM to 10 μM or more, are added to the culture media of defined wells in the presence of an appropriate concentration of glutamate (preferably in the range of 1 nM to 1 μM).

Secondary messenger assays, amenable to high throughput screening analysis, are performed including but not limited to the measurement of intracellular levels of cAMP, intracellular inositol phosphate, intracellular diacylglycerol concentrations, arachinoid acid concentration or tyrosine kinase activity (as described above). For example, the human metabotropic glutamate receptor protein (hmGluR) activity, as measured in a cyclic AMP assay, is quantified by a radioimmunoassay as described above. Results are compared to the baseline level of human metabotropic glutamate receptor protein (hmGluR) activity obtained from recombinant cells according to the invention in the presence of glutamate but in the absence of added modulator compound. Wells showing at least 2 fold, preferably 5 fold, more preferably 10 fold and most preferably a 100 fold or more increase or decrease in human metabotropic glutamate receptor protein (hmGluR) activity as compared to the level of activity in the absence of modulator, are selected for further analysis. Other variations are also possible as are control cell populations for use in a high-throughput format.

Dosage and Mode of Administration

By way of example, a patient can be treated as follows by the administration of a modulator of human metabotropic glutamate receptor protein (hmGluR) (for example, an agonist, antagonist or an allosteric modulator identified by any one of the methods of the herein disclosed invention. A modulator of human metabotropic glutamate receptor protein (hmGluR) the invention can be administered to the patient, preferably in a biologically compatible solution or a pharmaceutically acceptable delivery vehicle, by ingestion, injection, inhalation or any number of other methods. The dosages administered will vary from patient to patient; a “therapeutically effective dose” can be determined, for example but not limited to, by the level of enhancement of function (e.g., as determined in a second messenger assay described herein). Monitoring glutamate binding will also enable one skilled in the art to select and adjust the dosages administered. The dosage of a modulator of human metabotropic glutamate receptor protein (hmGluR) of the invention may be repeated daily, weekly, monthly, yearly, or as considered appropriate by the treating physician.

Pharmaceutical Preparations of Identified Agents

After identifying certain test compounds as potentially useful modulating moieties i.e., receptor agonists, receptor antagonists or receptor potentiators, the practitioner of the subject assay will continue to test the efficacy and specificity of the selected compounds both in vitro and in vivo. Whether for subsequent in vivo testing, or for administration to an animal as an approved drug, agents identified in the subject assay can be formulated in pharmaceutical preparations for in vivo administration to an animal, preferably a human.

The compounds selected in the subject assay, or a pharmaceutically acceptable salt thereof, may accordingly be formulated for administration with a biologically acceptable medium, such as water, buffered saline, polyol (for example, glycerol, propylene glycol, liquid polyethylene glycol and the like) or suitable mixtures thereof. The optimum concentration of the active ingredient(s) in the chosen medium can be determined empirically, according to procedures well known to medicinal chemists. As used herein, “biologically acceptable medium” includes any and all solvents, dispersion media, and the like which may be appropriate for the desired route of administration of the pharmaceutical preparation. The use of such media for pharmaceutically active substances is known in the art. Except insofar as any conventional media or agent is incompatible with the activity of the compound, its use in the pharmaceutical preparation of the invention is contemplated. Suitable vehicles and their formulation inclusive of other proteins are described, for example, in the book Remington's Pharmaceutical Sciences (Remington's Pharmaceutical Sciences. Mack Publishing Company, Easton, Pa., USA 1985). These vehicles include injectable “deposit formulations”. Based on the above, such pharmaceutical formulations include, although not exclusively, solutions or freeze-dried powders of the compound in association with one or more pharmaceutically acceptable vehicles or diluents, and contained in buffered media at a suitable pH and isosmotic with physiological fluids. In preferred embodiment, the compound can be disposed in a sterile preparation for topical and/or systemic administration. In the case of freeze-dried preparations, supporting excipients such as, but not exclusively, mannitol or glycine may be used and appropriate buffered solutions of the desired volume will be provided so as to obtain adequate isotonic buffered solutions of the desired pH. Similar solutions may also be used for the pharmaceutical compositions of compounds in isotonic solutions of the desired volume and include, but not exclusively, the use of buffered saline solutions with phosphate or citrate at suitable concentrations so as to obtain at all times isotonic pharmaceutical preparations of the desired pH, (for example, neutral pH).

Pharmaceutical preparations for oral use can be obtained through combination of active compounds with solid excipient, optionally grinding a resulting mixture, and processing the mixture of granules, after adding suitable auxiliaries, if desired, to obtain tablets or dragee cores. Suitable excipients are carbohydrate or protein fillers such as sugars, including lactose, sucrose, mannitol, or sorbitol; starch from corn, wheat, rice, potato, or other plants; cellulose such as methyl cellulose, hydroxypropylmethyl-cellulose, or sodium carboxymethyl cellulose; and gums including arabic and tragacanth; and proteins such as gelatin and collagen. If desired, disintegrating or solubilizing agents may be added, such as the cross-linked polyvinyl pyrrolidone, agar, alginic acid, or a salt thereof, such as sodium alginate.

### EXEMPLIFICATION

The invention now being generally described will be more readily understood by reference to the following examples, which are included merely for purposes of illustration of certain aspects and embodiments of the present invention and are not intended to limit the invention.

### Example 1

CLONING OF mGLAST

The full-length cDNA of mouse GLAST was isolated by PCR from a Marathon-Ready mouse brain cDNA library (Clontech, Palo Alto, CA). A 1725 bp fragment was amplified by PCR using Pfu Turbo DNA Polymerase with the following cycling conditions: a 2 min pre-incubation at 95° C., followed by 35 cycles of 95° C. for 30 sec., 56° C. for 30 sec., and 72° C. for 3 min. This fragment was obtained using the N-terminal primer, (5′GCCACCATGACCAAAAGCAACGGAGA 3′) containing an optimized Kozak sequence (GCCACC) and the C-terminal primer (5′ GAAAGTGAGCCCAGGGAGAT 3′) resulting in the inclusion of 80 basepairs of 3′ untranslated region. The amplified fragment was cloned into the PCR-Blunt II-Topo vector (Invitrogen, Carlsbad, Calif.). Following confirmation of the DNA sequence, the entire coding sequence of the gene was excised by EcoRI and sub-cloned into the mammalian expression vector pIRESneo2 (Invitrogen, Carlsbad, Calif.).

Generation of Stable Cell Lines Co-Eexpressing mGluR With mGLAST:

pCMV-T7-hmGluR5 (Daggett, LP et al. (1995).Neuropharmacology 34(8): 871-86) was digested with HpaI and EcoRI (New England Biolabs) and the isolated hmGluR5 fragment was subcloned into pIRESpuro2 (Clontech) digested with HpaI and EcoRI (New England Biolabs) and dephosphorylated with shrimp alkaline phosphatase (Roche). Ligations were transformed into competent DH5□ cells (Gibco BRL) and transformants were screened for hmGluR5 insertion by restriction digest with HpaI and EcoRI. Plasmid DNA was isolated by Qiagen Maxi Preps (Qiagen). Stable cell lines were established after transfection of CHONFAT-β-lactamase or Gqi5CHONFAT-β-lactamase with Lipofectamine 2000 (GIBCO) and drug selection with 10 μg/mL puromycin (Clontech). Positive expression was determined by measuring Ca²⁺ flux using a FLIPR₃₈₄, fluorometric imaging plate reader (Molecular Devices, Sunnyvale USA). Cells were grown in Dulbecco's modified medium (Gibco 11960) containing 10% dialyzed fetal bovine serum (Gibco 26400), 2 mM L-glutamine (Gibco 25030), 100 units/ml penicillin/streptomycin (Gibco 15070), non-essential amino acids (Gibco 11120), 25 mM HEPES (Gibco 15630), 55 μM β-mercaptoethanol (Gibco/BRL 21985) and 10 μg/ml puromycin (Clontech 8052-2). A double stable cell line was generated co-expressimg mGluR5 with mGLAST through transfection of pIRESneomGLAST into stable clones selected to express mGluR5 and drug selection with 1 mg/mL G418 (Gibco). Positive expression of GLAST was measured with a glutamate uptake assay.

Sodium Dependent [³H]Glutamate Uptake Assay:

Cells were plated in 96-well poly-D-lysine coated plates (Becton Dickinson) at a density of 80,000 cells per well 24 hours before assay and grown in normal growth media. The media was changed to glutamate/glutamine-free media and incubated for four hours prior to assay. Cells were washed two times in pre-warmed NaCl assay buffer (5 mM Tris, 10 mM HEPES, 140 mM NaCl, 2.5 mM KCl, 1.2 mM MgCl₂, 1.2 mM CaCl₂, 1.2 mM K₂HPO₄, 10 mM dextrose) or choline assay buffer (5 mM Tris, 10 mM HEPES, 140 mM choline, 2.5 mM KCl, 1.2 mM MgCl₂, 1.2 mM CaCl₂, 1.2 mM K₂HPO₄, 10 mM dextrose). For assay, [³H]glutamate (NEN-395, 22.5 Ci/mmole) was added to a final concentration of 500 nM and incubated for 5 minutes at 37° C., 5% CO₂. The reaction was stopped by washing the plate two times with cold choline assay buffer. Cells were lysed with 50 μL of 0.1N NaOH with shaking for 30 minutes. Twenty microliters of the lysate was transferred into a 96-well Optiplate (Packard) plate and 80 μL of Microscint-20 (Packard) was added. The plate was sealed, mixed and counted on a Beckman TopCount. Protein concentration was determined on 15 μL of the lysate. Na⁺-dependent [³H]glutamate uptake was determined by subtracting the total count in choline assay buffer from total in NaCl assay buffer,

Fluorometric Imaging Plate Reader (FLIPR) Assay:

CHO cells expressing mGluR5 receptors (mGluR5 CHO cells) were plated in clear-bottomed, poly-D-lysine coated 384-well plates (Becton-Dickinson 35-6663 Franklin Lakes USA) in glutamate/glutamine-free medium using a Multidrop 384 cell dispenser (Thermo Labsystems, Franklin USA). The plated cells were grown overnight at 37° C. in the presence of 6% CO₂. The following day, the cells were washed with 3×100 μl assay buffer (Hanks Balanced Salt Solution (Gibco 14025) containing 20 mM HEPES (Gibco 15630), 2.5 mM probenicid (Sigma P-8761), and 0.1% bovine serum albumin (Sigma) using an Embla cell washer (Skatron, Lier Norway). The cells were incubated with 1 μM Fluo-4AM (Molecular Probes) for 1 h at 37° C. and 6% CO₂. The extracellular dye was removed by washing as described above. Ca²⁺ flux was measured using FLIPR₃₈₄, fluorometric imaging plate reader (Molecular Devices, Sunnyvale USA). For potency determination, the cells were pre-incubated with various concentrations of compound for 5 min and then stimulated for 3 min with either an EC₂₀ or EC₅₀ concentration of agonist (i.e. glutamate) for potentiation measurements or antagonist measurements, respectively.

Reporter Gene Assay:

Aurora transcription based reporter cell lines were used to develop reporter gene assays for the mGluRs. The cell line, CHONFAT-β-lactamase, reports signaling through G_{q}-coupled receptors via an increase in intracellular calcium. The β-lactamase gene is under the transcriptional control of the nuclear factor of activated T-cells (N-FAT) promoter (reporting increased intracellular calcium). A second cell line Gqi5CHONFAT was utilized for Gi coupled mGluRs. This cell line contains a promiscuous G-protein that promotes coupling to a G_{q} signal transduction cascade. This results in the release of intracellular calcium and activation of NFAT. The production of β-lactamase resulting from the downstream signaling events of receptor activation is detected by loading cells with a fluorescent dye, CCF2-AM, a substrate cleavable by the β-lactamase enzyme. In its cleaved state, the substrate will fluoresce blue (EM460) when stimulated with UV light (395 nm). Noncleaved, the intact dye fluoresces green (EM530) (Science 1998, 279:84-88). The ratio of blue to green cells (Em460/530) is determined as a measure of signal transduction, with cells that have transduced a signal being blue and those that have not green.

Cells were plated at 80,000 cells per well in poly-D-lysine coated black clear bottom plates (Becton Dickinson) and grown in glutamate/glutamine free media (DMEM, -glutamine, 10% dialyzed fetal calf serum, 100 units/ml penicillin/streptomycin, 0.1 mM non-essential amino acids, 1 mM sodium pyruvate, 25 mM HEPES, 0.25 mg/mL zeocin, 10 μg/ml puromycin (Clontech 8052-2) and 1 mg/mL G418 for cell lines co-expressing mGluRs and mGLAST. All media was from GIBCO unless specified. Cells were grown overnight at 5% CO₂, 37° C. The next day, media was aspirated and the cells were washed twice with serum-free DMEM and replaced with 100 μL of assay media (DMEM, glutamine free), 0.1% BSA, 25 mM HEPES. Test ligands were added 10 min prior to addition of agonist, After a 4 hour incubation at 37° C., 5% CO₂, CCF2-AM loading dye was added. Plates were read 45-60 minutes later on a fluorescence plate reader (excitation 405±10 nm, emission 460±20 nm for blue; 530±15 nm. Wells containing dye and no cells were used to subtract background values.

1. A process for determining whether a test compound specifically binds to and modulates one or more cellular receptor proteins, comprising the steps of:
(a) contacting test cells co-expressing (i) a cell surface receptor protein and (ii) a neurotransmitter transport protein specific for a ligand of said cell surface receptor protein, wherein said cell produces a second messenger response upon activation of the surface protein, with (i) the test compound and a second compound known to activate the metabotropic glutamate receptor under conditions suitable for activation of the cell surface receptor protein and (ii) a control cell population wherein said cell do not express a functional cell surface receptor protein,
(b) measuring the second messenger response in the control cell population to obtain a first value and in the test cell population to obtain a second value,
(c) comparing the values obtained in (b), wherein if the second value is greater than the first value indicates that the test compound activates the target cell surface receptor protein, and wherein if the first value is greater than the second value indicating that the test compound inhibits activation of the target cell surface receptor protein.

2. The process according to claim 1, wherein said target cell surface receptor protein is a human metabotropic glutamate receptor selected from the group consisting of mGluR-1, -2, -3, -4, -5.,-6,-7 and -8.

3. The process of claim 1, wherein the second messenger response comprises change in intracellular calcium levels and the change in second messenger response is an increase in the measure of intracellular calcium in the test cell population relative to the control cell population.

4. The process of claim 1, wherein the second messenger response comprises release of inositol phosphate and the change in second messenger response is an increase in the level of inositol phosphate in the test cell population relative to the control cell population.

5. The process of claim 1, wherein the second messenger response comprises release of cyclic AMP (cAMP) and the change in second messenger response is a decrease in the level of cAMP in the test cell population relative to the control cell population.

6. The process according to claim 1, wherein said glutamate transporter protein is a murine glutamate transporter protein.

7. The process according to claim 1, wherein said metabotropic glutamate receptor is a human metabotropic glutamate receptor.

8. A mammalian cell-based assay for the profiling and screening of putative modulators of one or more human metabotropic glutamate receptor proteins, comprising:
(a) contacting a cell population comprising a plurality of cells co-expressing at least one functional human metabotropic glutamate receptor subtype or a variant, fragment or functional equivalent thereof and a functional non-human neurotransmitter transport protein or a variant, fragment or functional equivalent thereof specific for a ligand of said receptor and preloaded with a membrane potential fluorescent dye with (i) at least one modulating moiety whose ability to modulate the activity of the receptor protein is sought to be determined and (ii) a known agonist of said receptor protein; and
(b) monitoring changes in fluorescence of the cells in the presence of the modulating moiety compared to changes in the absence of the modulating moiety to determine extent of human metabotropic glutamate receptor modulation.

9. The assay method of claim 8 in which the test cell is selected from the group consisting of MOCK, HEK293, HEK293T, BHK, COS, NIH3T3, Swiss3T3 and CHO.

10. The assay method of claim 8 in which the known agonist is added prior to, concurrently or after addition of the modulating moiety.

11. (canceled)

12. The assay method of claim 8 in which a said method is used to identify a compound as one which particularly modulates an mGluR activity based on a detectable change in fluorescence.

13. (canceled)

14. The assay method of claim 8 wherein said cells are loaded with a membrane potential dye that allows for changes in fluorescence to be detected.

15. The assay method of claim 8 wherein said cell expresses at least one human mGluR subtype.

16-18. (canceled)

19. The assay of claim 8 wherein said fluorescent dye is a calcium-sensitive fluorescent dye.

20. (canceled)

21. The assay of claim 8 wherein said instrument is one of a fluorescence plate reader (FLIPR) or is a voltage imaging plate reader (VIPR).

22. (canceled)

23. A method for identifying a modulator of one or more mammalian metabotropic glutamate receptor proteins, comprising:
(a) providing a cell population containing a plurality of recombinant test cells modified to contain the DNA of (i) a mammalian glutamate receptor subtype or a variant, fragment or functional equivalent thereof which is operably linked to control sequences for expression whose activation can be coupled to Ca²⁺ signaling pathway, and (ii) a functional non-human neurotransmitter protein or a variant, fragment or functional equivalent thereof specific for a ligand of said receptor;
(b) providing at least one compound or modulating moiety whose ability to modulate the activity of a metabotropic glutamate receptor protein is sought to be determined,
(c) incubating or contacting the cell population with the modulating moiety and a calcium sensitive-fluorescent dye to form a first mixture;
(d) measuring fluorescence from the calcium-sensitive fluorescent dye in the first mixture in a fluorometric imaging plate reader (FLIPR) to obtain a first value;
(e) repeating steps a-c except to obtain a second mixture except that the cell population comprises cells that do not express a functional metabotropic glutamate receptor protein;
(f) measuring fluorescence from the calcium-sensitive fluorescent dye in the second mixture in a fluorometric imaging plate reader (FLIPR) to obtain a second value;
(g) comparing the fluorescence measurement from d) with the fluorescence measurement of f), wherein if the first value in the first mixture is greater than that of the second mixture, then said at least one test modulating moiety is a positive modulator of the metabotropic glutamate receptor protein.

24. A method for identifying a metabotropic glutamate negative allosteric modulator of one or more metabotropic glutamate receptor subtypes having inhibitory activity, said method comprising the steps of
(a) exposing a cell population comprising cells co-expressing at least one functional metabotropic glutamate receptor subtype or a variant, fragment or functional equivalent thereof and a functional non-human neurotransmitter transport protein or a variant, fragment or functional equivalent thereof to the candidate agent in the presence of a known metabotropic glutamate agonist, wherein said cells produces a second messenger response upon activation of the metabotropic glutamate receptor subtype, under conditions and for a time sufficient to allow interaction of the agonist with the receptor and an associated activation of the metabotropic glutamate receptor, and
(b) detecting an inhibition of the second messenger response by the agonist resulting from the interaction of the candidate agent with the metabotropic glutamate receptor subtype, relative to the second messenger response induced by the glutamate agonist alone, and therefrom determining the presence of a glutamate allosteric modulator having antagonist-like activity.

25. The process of claim 24, wherein said test cell constitutively expresses the mGluR5 receptor subtype.

26. (canceled)

27. A method for identifying a metabotropic glutamate positive allosteric modulator of one or more metabotropic glutamate receptor subtypes having antagonistic activity, said method comprising the steps of
(a) exposing a cell population comprising cells co-expressing at least one functional metabotropic glutamate receptor subtype or a variant, fragment or functional equivalent thereof and a functional non-human neurotransmitter transport protein or a variant, fragment or functional equivalent thereof to the candidate agent in the presence of a known metabotropic glutamate agonist, wherein said cells produces a second messenger response upon activation of the metabotropic glutamate receptor subtype, under conditions and for a time sufficient to allow interaction of the agonist with the receptor and an associated activation of the metabotropic glutamate receptor, and
(b) detecting activation of the second messenger response by the agonist resulting from the interaction of the candidate agent with the metabotropic glutamate receptor subtype, relative to the second messenger response induced by the glutamate agonist alone, and therefrom determining the presence of a metabotropic glutamate allosteric modulator having agonist-like or activating activity.

28. A process for screening a candidate agent for the ability of the candidate agent to positively modulate one or more metabotropic glutamate receptor subtype mediated signal transmission pathway in a mammalian cell comprising:
(a) incubating a test cell population with a candidate agent whose ability to modulate the second messenger activity of the receptor is sought to be determined, wherein said test cell: population is characterized as comprising a plurality of cell co-expresses a functional metabotropic glutamate receptor subtype glutamate receptor subtype or a variant, fragment or functional equivalent thereof and a functional non-human glutamate transport protein in or a variant, fragment or functional equivalent thereof; and wherein said cells are transformed with a recombinant DNA molecule comprising a reporter gene operably linked to a regulatory sequence which responds to a change in intracellular concentration of one or more second messenger substances of a metabotropic glutamate receptor-mediated signal transmission pathway, wherein said response is a change in the expression of a reporter gene in said test mammalian cell, said expression being indicated by production of a reporter gene product;
(b) measuring the concentration of the reporter gene product in the test cell population
(c) comparing the concentration of the reporter gene product in said test cell to the concentration of said reporter gene product in a control cell population which are identical to the test cells except that the cells of the control cell population do not express a functional metabotropic glutamate receptor subtype; wherein a higher concentration in said test cell relative to the concentration in said control cell indicates that the test substance has activating activity on said signal transmission pathway, and wherein a lower concentration in said test cell relative to the concentration in said control cell indicates that said test substance has inhibitory activity on said signal transmission pathway.

29. The process according to claim 27, wherein said recombinant DNA comprises a regulatory sequence which responds to one of (i) a change in concentration of intracellular calcium brought about by modulation of said receptor or (ii) a change in concentration of cyclic AMP.

30. (canceled)

31. A method for identifying candidate therapeutic agents for the treatment of a metabotropic glutamate receptor mediated disorder, comprising:
(a) incubating a cell population comprising a plurality of cells co-expressing on a surface thereof at least one metabotropic glutamate receptor subtype glutamate receptor subtype or a variant, fragment or functional equivalent thereof and a functional non-human neurotransmitter transport protein or a variant, fragment or functional equivalent thereof with a test compound and a known mGluR agonist, wherein said cells comprises a reporter construct responsive to a change in one of or more second messenger substances, and wherein said reporter construct comprises a nucleotide sequence encoding a reporter protein operably linked to a responsive regulatory element responsive to a change in a second messenger resulting from activation of said receptor protein,
(b) measuring the expression of the reporter gene product in the presence of the test compound and the agonist and comparing the value to that obtained in the absence of the test compound;
(c) selecting a test compound that decreases the expression of the reporter gene product in the presence of the agonist compared to the expression of the reporter gene product in the presence of the test compound alone; and
(d) identifying the selected test compound as a candidate therapeutic agent for treatment of a neurodegenerative disorder.

32. The method of claim 31, wherein said reporter coding sequence is selected from the group consisting of a luciferase, green fluorescent protein, β-lactamase, β-galactosidase, β-glucuronidase; Alkaline phosphatase; blue fluorescent protein, and chloramphenicol acetyl transferase.

33. A method for identifying potential allosteric modulators of a mammalian metabotropic glutamate receptor, comprising:
(a) incubating a test cell population comprising a plurality of cells co-expressing on a surface thereof at least one metabotropic glutamate receptor subtype glutamate receptor subtype or a variant, fragment or functional equivalent thereof and a functional non-human neurotransmitter transport protein or a variant, fragment or functional equivalent thereof with a known amount of a known mGluR agonist, wherein said cells comprises a reporter construct responsive to a change in one of or more second messenger substances, and wherein said reporter construct comprises a nucleotide sequence encoding a reporter protein operably linked to a responsive regulatory element responsive to a change in a second messenger resulting from activation of said receptor protein,
(b) incubating a control cell population comprising a plurality of cells co-expressing on a surface thereof at least one metabotropic glutamate receptor subtype and a non-human neurotransmitter transport protein specific for a ligand of said receptor with a known amount of a known mGluR agonist, wherein said cells comprises a reporter construct responsive to a change in one of or more second messenger substances, and wherein said reporter construct comprises a nucleotide sequence encoding a reporter protein operably linked to a responsive regulatory element responsive to a change in a second messenger resulting from activation of said receptor protein,
(c) measuring the expression of the reporter gene product in the presence of the known agonist and comparing the value to that obtained in the absence of the known agonist but in the presence of a test compound alone;
(d) selecting a test compound that increases or decreases the expression of the reporter gene product in the presence of the test compound alone compared to the expression of the reporter gene product in the presence of the agonist alone; and
(e) identifying the selected test compound as a candidate therapeutic agent for the treatment of a neurodegenerative disease mediated by a metabotropic glutamate receptor subtype and which is susceptible to allosteric modulation by said therapeutic agent.

34. Host cells transformed with a nucleic acid construct under conditions favoring expression of at least one metabotropic glutamate receptor protein on a surface of said cells and a non-human neurotransmitter transport protein specific for a ligand of said receptor protein.

35. A process for determining whether a candidate agent is a metabotropic glutamate receptor antagonist which comprises contacting cells co-expressing a functional metabotropic glutamate receptor and a glutamate transporter protein cells with the candidate agent under conditions favoring activation of a functional metabotropic glutamate receptor, with the proviso that said cells co-express a functional glutamate transporter, and detecting any decrease in metabotropic glutamate receptor activity, as indicating that the candidate agent is a metabotropic glutamate receptor antagonist.

36. A method of screening a plurality of test compounds to identify a candidate compound which inhibits the activation of one or more human metabotropic glutamate receptor subtypes, said method comprising the step of
(a) contacting cells co-expressing at least one metabotropic glutamate receptor subtype and a neurotransmitter transport protein specific for a ligand of said metabotropic glutamate receptor subtype, wherein said cells produce a second messenger response upon activation of the metabotropic glutamate receptor, with the plurality of test compounds in the presence of a known metabotropic glutamate receptor agonist under conditions suitable for activation of the metabotropic glutamate receptor, and
(b) determining whether the extent or amount of activation of metabotropic glutamate receptor is reduced in the presence of one or more of the test compounds, relative to the extent or amount of activation of the metabotropic glutamate receptor in the absence of said one or more test compounds, and if so,
(c) separately determining whether each such compound inhibits activation of metabotropic glutamate receptor for each compound in the plurality of compounds, so as to identify any compound in such plurality of compounds which inhibits the activation of the metabotropic glutamate receptor.

37. A process for determining whether a candidate agent is a metabotropic glutamate receptor agonist which comprises contacting a control cell population comprising cells that do not express a functional metabotropic glutamate receptor protein and a test cell population comprising a plurality of cell co-transfected with nucleic acid encoding a metabotropic glutamate receptor under conditions favoring expression of the metabotropic glutamate receptor on a surface of said transfected cells and a functional glutamate transporter protein, with the candidate agent under conditions favoring activation of the metabotropic glutamate receptor and detecting any increase in human metabotropic glutamate receptor activity relative to a control cell population as indicating that the candidate agent is a metabotropic glutamate receptor agonist.

38. A process for determining whether a chemical compound specifically binds to and activates one or more metabotropic glutamate receptor subtypes, which comprises contacting cells producing a second messenger response and expressing on their cell surface at least one metabotropic glutamate receptor subtype, wherein such cells do not normally express the metabotropic glutamate receptor, with the chemical compound under conditions suitable for activation of the human metabotropic glutamate receptor and measuring the second messenger response in the presence and in the absence of the chemical compound, wherein a change in the second messenger response in the presence of the chemical compound indicating that the compound activates the metabotropic glutamate receptor subtype, with the provisio that said cell also express a glutamate transporter protein specific for a ligand bound by said metabotropic glutamate receptor subtype.
